(19) United States
(12) Patent Application Publication
(10) Pub. No.: US 2025/0201420 A1
(43) Pub. Date: Jun. 19, 2025

(54) SYSTEM AND METHOD FOR COMPREHENSIVE DIGITAL PLATFORM FOR MENTAL HEALTH ASSESSMENT, INTERVENTION, AND OUTCOMES TRACKING

(71) Applicant: AIBERRY, INC., BELLEVUE, WA (US)

(72) Inventors: EZRA MORSE, QUALICUM BEACH (CA); LINDA S. CHUNG, BELLEVUE, WA (US); JASON DEE SHUMAKE, AUSTIN, TX (US); LIOR AUSLANDER, BELLEVUE, WA (US); OVIDIU ROMAN, TACOMA, WA (US); HASSAN UGAIL, ILKLEY (GB); ALI AHMED M. ELMAHMUDI, BRADFORD (GB); ALIYU ABUKAKAR, BRADFORD (GB); AHMED AMAN IBRAHIM, MALE (MV); HONG PENGFEI, SINGAPORE (SG); MICHAEL COLLINS MULLARKEY, DENVER, CO (US); MATTHEW DOMINIC MATZE, TAMPA, FL (US); MONA VANA SCHREIBER, KIRKLAND, WA (US)

(73) Assignee: AIBERRY, INC., BELLEVUE, WA (US)

(21) Appl. No.: 18/959,400

(22) Filed: Nov. 25, 2024

Related U.S. Application Data
(63) Continuation-in-part of application No. PCT/US2023/024211, filed on Jun. 1, 2023.
(63) Continuation-in-part of application No. PCT/US2023/024289, filed on Jun. 2, 2023.
(63) Continuation-in-part of application No. PCT/US2023/024260, filed on Jun. 2, 2023.
(60) Provisional application No. 63/604,785, filed on Nov. 30, 2023.
(60) Provisional application No. 63/604,803, filed on Nov. 30, 2023.
(60) Provisional application No. 63/604,823, filed on Nov. 30, 2023.
(60) Provisional application No. 63/604,828, filed on Nov. 30, 2023.
(60) Provisional application No. 63/604,834, filed on Nov. 30, 2023.
(60) Provisional application No. 63/604,840, filed on Nov. 30, 2023.

(51) Int. Cl. G16H 50/30; G16H 20/10

(52) U.S. Cl. CPC G16H 50/30; G16H 20/10

(57) ABSTRACT

A computer-implemented method includes receiving patient responses over a network from a recorded screening interview, utilizing a custom Large Language Model (LLM) to generate transcriptions and insights from the audio, performing video sentiment analysis to assess emotional states, and based on these data, generating an AI model to predict risk levels for various mental health conditions. The method further comprises presenting personalized questions based on previous screening insights, creating longitudinal summaries of patient histories, continuously improving the AI models through reinforcement learning, and integrating a recommendation engine to suggest targeted interventions. Additionally, the method includes tracking outcomes over time and enabling causal inference across multiple screenings, thereby providing a comprehensive platform for mental health assessment, intervention, and outcomes tracking.

### PRIORITY CLAIM

This application claims priority to and the benefit of U.S. Provisional Patent Application Ser. No. 63/604,785 filed Nov. 30, 2023, U.S. Provisional Patent Application Ser. No. 63/604,803 filed Nov. 30, 2023, U.S. Provisional Patent Application Ser. No. 63/604,823 filed Nov. 30, 2023, U.S. Provisional Patent Application Ser. No. 63/604,828 filed Nov. 30, 2023, U.S. Provisional Patent Application Ser. No. 63/604,834 filed Nov. 30, 2023, and U.S. Provisional Patent Application Ser. No. 63/604,840 filed Nov. 30, 2023. This application is a Continuation-in-Part under 35 U.S.C. §§ 120 and 365 (c) of International Application No. PCT/US2023/024211 filed Jun. 1, 2023, International Application Serial No. PCT/US2023/024289 and International Application Serial No. PCT/US2023/024260, filed Jun. 2, 2023, the contents of each are hereby incorporated by reference in their entirety as if fully set forth herein.

### COPYRIGHT NOTICE

This disclosure is protected under United States and/or International Copyright Laws. @ 2024 AIBERRY, INC. All Rights Reserved. A portion of the disclosure of this patent document contains material which is subject to copyright protection. The copyright owner has no objection to the facsimile reproduction by anyone of the patent document or the patent disclosure, as it appears in the Patent and/or Trademark Office patent file or records, but otherwise reserves all copyrights whatsoever.

### BACKGROUND

Many mental health disorders are intertwined with each other and with cognitive, social, and environmental risk factors. For example, there is a strong correlation across Depression, Anxiety, Suicide Risk, Stress, Negative Self View, and Social Isolation. Yet for all such disorders, the prior art standard of care for psychological measurement is administration of disparate Likert-scale questionnaires, which provide an unnatural way for humans to express symptomatology and require a rare combination of a high degree of education, self-insight, and verbal ability to provide accurate self-assessment. Moreover, even when symptoms are described well by a multiple-choice questionnaire, they may or may not indicate an underlying mental health condition, depending on the most likely cause of the symptom (e.g., loss of sleep due to anxiety vs. being woken up by an infant). Further, these questionnaires all use different ranges on their scales, creating challenges when comparing scores for different conditions. The result of this ecosystem are unreliable measurements with problematic adherence, thereby complicating clinician's abilities to track conditions over time, to identify trailing or leading precipitating factors of mental health disorders, and to deliver proactive interventions.

In embodiments of our invention, artificial Intelligence and other technological innovations bring new capabilities to the mental health field, such as the ability to more accurately assess disorders from a patient's spoken description of their symptoms. These new capabilities of embodiments of our invention create a more engaging and naturalistic means of standardized mental health assessment that reduces barriers to reproducible measurement of mental health outcomes. In coordination, the embodiments of our invention address the accessibility barriers and measurement deficiencies of prior art form questionnaires, making it easier to effectively screen for risk factors and monitor the efficacy of treatments for the most common mental health conditions.

### BRIEF DESCRIPTION OF THE DRAWING FIGURES

FIGS. 1A-1B illustrate one embodiment of the present invention;

FIG. 2 illustrates another embodiment of the present invention showing a process illustration for creating the custom Large Language Model (LLM);

FIG. 3 illustrates the screening flow process (the flow from conducting a screening to getting the screening outputs (score, transcript, transcript and summary) in one embodiment of the invention;

FIG. 4 illustrates some of the uses for personalized screening questions based off past screening insights and notes in yet another embodiment of the invention;

FIGS. 5A-5E illustrate one embodiment of stress and burnout visual scales designed to correspond to specific questions asked in yet another embodiment of the invention;

FIG. 6 illustrates how the Stress and Burnout forms can assign tasks to patients via self-assessment capability in yet another embodiment of the invention;

FIG. 7 shows how the visual scale can be incorporated into a Burnout digital form;

FIG. 8 illustrates an embodiment of the invention in which the invention analyzes the participant's response and decides what the next question should be;

FIGS. 9A and 9B illustrate a Multimodal Stress and Burnout screening according to an embodiment;

FIGS. 10A-10D illustrates an example for mapping insights to external library resources in yet another embodiment of the invention;

FIG. 11 illustrates screening insights in one embodiment of the invention;

FIGS. 12 and 13 illustrate screening insights histograms in further embodiments of the invention;

FIG. 14 illustrates four different screening types (Depression, Self-View, Stress and Burnout, Anxiety) on a single graph showing a normalized histogram for various risk scores in one embodiment of the invention;

FIG. 15 illustrates the reliable change analytics in one embodiment of the invention;

FIG. 16 illustrates how insights created from an initial screening can be used in a subsequent screening to create personalized questions;

FIG. 17 illustrates the process of concatenating a patient's LLM notes and insights from their history, and inputting it into another custom LLM to create a longitudinal summary;

FIG. 18 illustrates how one or more embodiments support collecting clinical data continuously with Reinforcement Learning through Human Feedback to incrementally fine-tune artificial intelligence models;

FIG. 19 is a visualization of the Multimodal (Text/Audio/Video) Stress and Burnout inference pipeline;

FIG. 20 illustrates the usage of Retrieval Augmented Generative searching to intelligently recommend targeted mental health resources from internal or external libraries;

FIG. 21 illustrates the ability to make causal inferences by graphing annotations of external events overlaid on top of a cohort's outcomes over time.

### DETAILED DESCRIPTION

The following embodiments comprise an inventive digital health software platform designed to support mental health assessment, treatment, and outcomes tracking. At the core of the platform, Artificial Intelligence (AI) is leveraged to allow patients to verbally describe their conditions in natural language; such conversations are converted to insightful notes and summaries that provide clinicians with unprecedented valuable patient insights and risk scores. The platform also includes innovative visual scales that allow patients to intuitively self-report on their levels of stress and burnout. Beyond assessment, the platform leverages these screening insights to deliver personalized recommendations for relevant mental health resources and content. The platform can also detect relationships between different mental health risk factors, enabling preventative interventions. Finally, the platform tracks mental health outcome metrics over time, demonstrating the effectiveness of treatments for individual patients and patient populations. Together, these features create a powerful digital ecosystem for developing and deploying novel artificial intelligence (AI) models and supporting holistic patient mental health by leveraging such AI technology.

### Large Language Model Based Screening Notes Generator

One or more embodiments include a custom Large Language Model (LLM), that takes a conversational screening transcript as an input and generates specific insights and notes that are useful and relevant to clinicians.

For background, LLMs are a type of artificial intelligence (AI) that can process and generate human-like text; they accept a string of text as input and they typically generate human-like content in response to the given text. These models are built on Google™'s Transformer technology (“Attention Is All You Need”), which are mathematical functions that convert a series of numbers to a multi-dimensional “vector”, such that a vector is defined as a direction with a magnitude. For an example of a 3-dimensional vector, if a person is sitting at a dining table and they were looking for a plate, a 1-foot vector pointing away from their chest would accurately describe the location of the plate. If an extra dimension is added to the example, it could be assumed that the 4th dimension may refer to time, and it may be difficult to visualize how such a vector might operate; perhaps it would point to the precise frame in time in which the person was sitting in front of the table.

In contrast to the given example, Transformers do not operate in 3-dimensional space, but may instead produce up to 4096-dimensional vectors. They do this by first converting the words in a sentence to discrete numeric tokens, and then for each numeric token, they compute a new vector from the last one; every word builds on the previous vector and has the ability to change the direction and magnitude contained therein. As a result, semantically similar sentences typically end up in the same vector space. For example, “I am doing great” and “I am doing very well” will be clustered closely together in their vector space. Similarly-referring to the previous real-world example—vectors of “a plate”, “a spoon” and “the dining table” would all point to the same 3-dimensional space compared to vectors pointing to “the moon” or “the sun”, relative to a person sitting at a table.

Transformers can be trained such that their resultant vectors can then be used to predict the next most probable numeric token in a series of tokens, as reflected in the training set. For example, “the cat was” could be converted to a series of 3 numeric tokens and vectorized by the transformer. At which point, the vector may be used to predict with 66% probability that “purring” is the most likely token if it was seen as such in 2 out of 3 relevant training documents (with “relevance” implying that the training documents likely share the same vector space as the current sequence). At which point, the new predicted token would be added to the sequence, forming the sentence “the cat was purring”. The additional “purring” token can then be used to update the previous vector, and thereby make another prediction until a complete sentence or paragraph is formed. Transformers trained on custom datasets with the intention to generate human-like content are called Generative Pre-trained Transformers (GPT), which are a type of LLM. GPT architecture is one option or example of what many of the following embodiments may be based on or utilized, and it should be assumed in the usage of the term Large Language Model (LLM), GPT technology or similar technology has been used to train and infer from the model.

These LLMs are trained on vast amounts of human generated content and consider billions of parameters (aka, variables) in their computations. Thereby, these models are very adept at predicting the most probable word(s) humans would produce next in a sequence of words, and thus, can generate content that is practically indistinguishable from content generated by humans. These LLMs can also be “fine-tuned”, a process in which a custom model is produced from a base model that slightly modifies the original model's internal mathematical formulas (or weights) to predict a very different set of words. Considering the previous example, a fine-tuning training set could contain many training examples of “the cat was sleeping”. As such, the resultant fine-tuned model may examine the string “the cat was” and predict that “sleeping” is far more probable than “purring”.

Through the process of “fine-tuning”, the base behavior of a LLM can be modified by having expert humans generate training examples for specific purposes. Many of the original behaviors of the LLM will remain intact (such as its ability to create conjunctions or pick the proper pronouns), but, for example, a model can be tuned to generate fictional prose, clinical diagnosis information, or even computer code. After fine-tuning, the natural behavior of a base model will be altered, and the result will be more similar to the human generated training material, creating the ability to modify a general-purpose LLM for very specialized purposes.

In a preferred embodiment, the fact that the model is trained on proprietary Aiberry data and hosted on Aiberry infrastructure ensures that this generative AI capability is well-bounded and predictable. This may be achieved through the fact that a specific custom and fine-tuned model is created and trained on specific inventive data, which is gathered in a clinical context in compliance with the Health Insurance Portability and Accountability Act of 1996 (HIPAA) (Public Law 104-191, see 42 U.S.C. § 1320d-2. As such, this preferred embodiment is not a public and open model but is rather a custom model. However, alternate embodiments of the invention may train the model on other proprietary or non-proprietary open or public data and hosted on other proprietary or non-proprietary open or public infrastructures. Similarly, alternate embodiments of the invention may feature generative AI capability with varying degrees of bounds and predictability.

Among the distinguishing features of one or more embodiments is that Aiberry owns the transcript data and has used qualified clinicians to create matching useful and relevant summaries for each of these transcripts by using the user interface of embodiments of the Aiberry invention. Each resultant training set contains an anonymized screening transcript, which is paired with a summary and insights that were manually written by a certified clinician for diagnostic purposes. While most Large Language Models are typically trained from publicly available datasets, and thus can be retrained by the public to reproduce the same model weights, Aiberry's LLM is based on this unique private training set of screening transcripts and clinician written summaries that cannot be replicated from publicly available data; at least because such transcripts are healthcare-related information protected by HIPAA, such information cannot readily be shared between separate entities. According to one or more embodiments, a unique capability is supported to both collect protected health information and use such proprietary data to train AI models internally.

As previously explained, LLMs are mathematical functions that predict the next most probable word (or token) in a given sequence of words; their probabilistic determinations are reflective of the training data used to create them. As such, an LLM trained to produce non-clinical text completions from non-clinical instruction input data will not be adept at predicting words that form relevant and useful sentences to clinicians about clinical transcripts. For example, a general-use LLM that evaluates a sequence of tokens such as “The crying man was” will likely simply predict “sad” as the next token with a high probability.

By training an LLM with summaries and insights written by actual clinicians, the resulting model weights can provide specific insights that would not be possible without this combination of proprietary data. For example, a clinician may see the aforementioned sequence of “The crying man was” and produce a diagnostic completion such as “suffering from Major Depressive Disorder”. By interaction with the embodiments of the Aiberry invention, the clinician provides their useful and relevant description in the “summary_text” field of the training data. Due to training on this clinically-relevant data, the resultant fine-tuned LLM will associate much higher probabilities for those clinical words that form useful and relevant sentences for this context, resulting in emergent clinical diagnostic behavior that is strictly unique to Aiberry's custom clinically-trained models. In alternate embodiments, the fine-tuning of the LLM may be based on training the model on other curated training data.

Once the training set is generated by the certified clinicians, the base-model then must be fine-tuned in order to have its behavior altered to emulate the desired clinical behavior modeled in the examples. According to one or more embodiments, fine-tuned models can be generated from the training examples using Python, an interpreted computer language adept at matrix multiplication, parallel processing and optimized graphical processing unit access, which makes it uniquely suitable for training models with billions of parameters. “APPENDIX 2-Fine-tuning Code” contains example code that processes this proprietary training data and uses it to fine-tune a custom LLM, such that it will produce summaries and insights relevant to clinicians (behavior that is defined by the aforementioned set).

Based on this technology, one or more embodiments include an alternative custom patient-facing LLM that displays relevant insights and notes to the patient instead of the practitioner. To create the patient-facing Notes Generator LLM, a similar training process is used as above (e.g. APPENDIX 2), but the training data is composed of the same clinical transcripts paired with informally written summaries and insights; such summaries and insights are again written by clinicians with the intent of sharing a diagnosis and insights with a hypothetical patient.

One embodiment of the invention includes a library of LLM summarizers that can be trained and tuned each to a well-defined purpose by simply pairing the same input data (such as clinical transcripts) with an intentionally crafted response written in the context of the desired purpose. For example, the sentence “My energy levels have been up and down” may be summarized to a practitioner as “The patient's energy levels have been fluctuating with no significant risk of an energy disorder”, while the patient notes model could summarize their response as “Varying levels of energy”. These summaries could come from the same base model that was fine-tuned into two different models that produce unique and disparate summaries from the same input transcript.

Based on this technology, one or more embodiments of the present invention include a custom LLM for each and every type of disorder screening (e.g., Anxiety, Self-View, etc.). To do so, the same training process is utilized as with the depression summaries, with the primary difference being that the training set consists of anonymous clinical transcripts for the other disorders paired with notes and insights created by clinicians to be useful and relevant to the target disorder.

One or more embodiments can include a more contextual screening interview, in which the system can use insights from past screenings to make the questions more personalized and relevant. For example, questions can be generated that refer to specific hobbies or people close or significant to the patient that were mentioned in the past by the patient.

### Capability 1—Generating a Clinically-Useful HIPAA Compliant Dataset

This capability describes the manner in which the invention collects, stores and outputs training sets for the purpose of training custom AI models that are useful for clinical diagnosis.

According to HIPAA legislation, Protected health information (PHI) in any form must be securely maintained, controlled and protected to prevent unauthorized access or disclosure. As such, PHI-such as interview transcripts or videos-cannot be shared between separate entities unless both are HIPAA compliant and they have entered into a contractual agreement concerning privacy, in accordance with the law (such as a Business Associate Agreement). Due to these stringent requirements, it is not legal to openly publish any dataset of mental health screenings that would be suitable for training AI models, because in doing so, it would breach the privacy of those patients involved. Hence, no such dataset publicly exists.

Furthermore, constructing a dataset with non-clinical data or mock data would fail to create a suitable dataset for training if the resultant models are intended to analyze HIPAA-compliant screenings collected in a clinical context.

The problem posed by a training set constructed from non-clinical data is that it is difficult to predict the manner in which patients conversationally respond to clinical questions asked during a screening. For example, Twitter™ responses have been used in many peer-reviewed studies to examine the detection of mood and suicidality from language. Due to the short-form nature of that medium, which had a character limit of 140 characters at times, such inputs do not reflect natural speech, and those models cannot generalize such that they are predictive from the spoken responses in a clinical interview. For the most useful predictions, models should be trained on inputs reflective of the expected live input, as gathered in similar contexts. Otherwise, a situation is created akin to feeding diesel fuel to an engine built to consume normal petrol, and as such, the engine's behavior will be unpredictable.

The problem posed with creating an artificial training set completely from mock data follows the previous problem of replicating live clinical conversations, but would likely also require human interaction to produce such a set. Being that Large Language Models may require hundreds to thousands of examples, manually creating a diverse, unbiased set reflective of clinical interviews would be an insurmountable feat for humans alone.

Being that such clinical datasets necessary for AI training cannot publicly exist or reasonably be created by prior art, a collection of embodiments-including, for example, those described in International Application Serial No. PCT/US23/24211-enables the capability to create and store such training sets that contain protected health information.

By means of a human interacting with a graphical interface according to one or more embodiments to conduct a bot-driven clinical mental health screening, multimodal (Text/Audio/Video) data is received over the network and stored in secured, encrypted HIPAA compliant storage. Importantly, this potential training data is collected through the exact same interactions with the invention as the screening data that will be later analyzed by the trained models, thereby ensuring that the training data is composed of spoken replies reflective of the expected clinical inputs.

According to an embodiment, such data is then anonymized through an automated computer process and made accessible for further processing through a graphical user interface. By interacting with such an interface, certified clinicians can then create training pairs in which the collected data is paired with useful and relevant summaries, insights and scores that the clinicians provide. These pairs create training sets, such that AI models can now be trained to take similar inputs from live screenings and generate summaries, insights and scores that are reflective of the clinical examples; such a training process will be explained in a later capability.

According to one or more embodiments, by means of interacting with a graphical interface, the datasets can be outputted to files in various formats-such as lined JSON—for conversational or completion AI training, and then transmitted to users over the network.

The combination of these embodiments result in unique, proprietary training sets composed of anonymized HIPAA-protected health information paired with clinician-validated useful information, which can be used to produce AI models that exhibit unprecedented clinically diagnostic behavior; such model behavior was not previously possible without such HIPAA compliant data collection and storage capabilities, as well as without interactive graphical interfaces that allow clinician to interact and create the training pairs that dictate such behavior.

Capability 2—Generating Screening Notes from a Screening Transcript

This capability describes the Aiberry invention's ability to accept a mental health screening transcript as an input and then use a custom LLM to generate key insights and summary notes that will help the clinician gain insight into the patient.

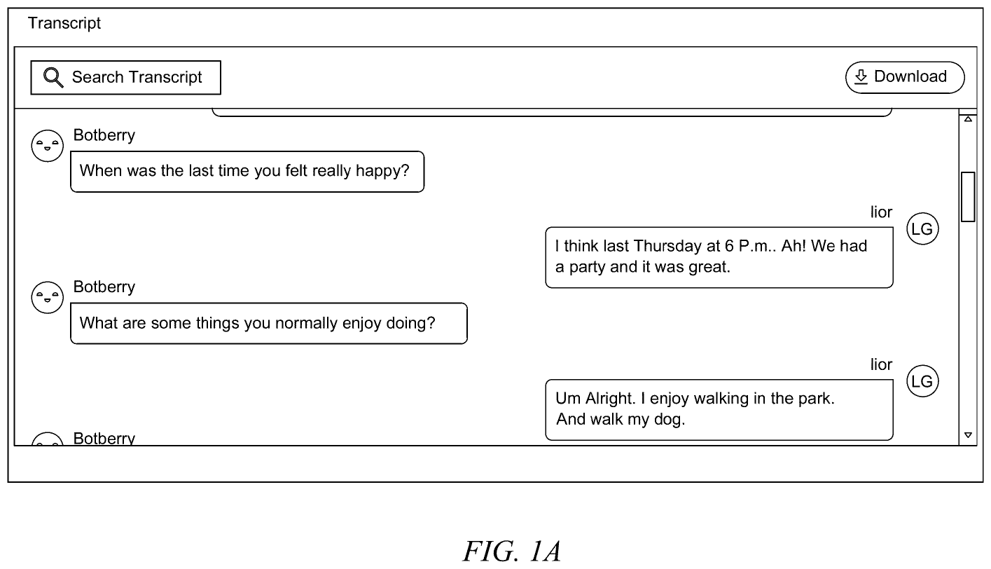

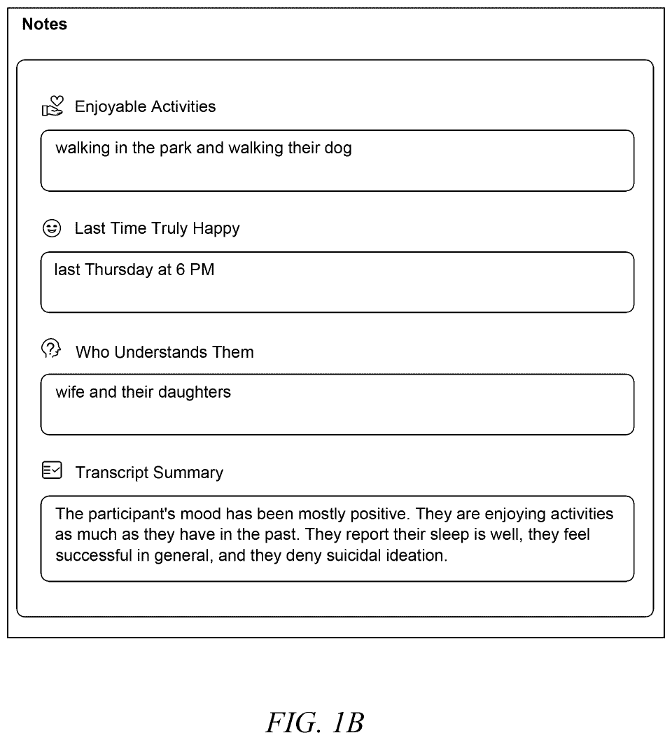

FIGS. 1A-1B shows a sample conversational screening transcript and how that is translated into the screening notes with specific insights around, for example, ‘Enjoyable Activities’.

From a technical perspective, the following are some steps followed in building a custom LLM:

1. Select an open-source, commercial license friendly base generative pre-trained transformer (GPT) large language model (LLM).

2. Create a proprietary clinical dataset, with ideal (from a clinician point of view) summaries and insights based on those transcripts. In one embodiment—as explained in “Capability 1-Generating a clinically-useful HIPAA compliant dataset”—we have collected anonymized data from our production system, and since Aiberry was proved to be clinically validated (https://pubmed.ncbi.nlm.nih.gov/38290584/), the data used in an embodiment from our production system is deemed as clinical dataset. For each clinical transcript, clinicians were tasked to manually create summaries and insights that are clinically useful and relevant.

3. Fine-tune the base LLM using the proprietary dataset to create a custom LLM, a process which is shown by example in APPENDIX 2. According to one or more embodiments, the LLM can be trained with such models, for example, as:

Flan-T5 from Google™, an open-source, sequence-to-sequence, large language model that can be also used commercially.

Llama 3.2 from Facebook™, a model that excels in tasks requiring image recognition and language processing. It can answer questions about images, generate descriptive captions, and even reason over complex visual data.

Once a model is chosen, a LORA fine-tuning process, as illustrated in APPENDIX 2. In this process, the dataset is split into a training set (80%) and a validation set (20%); all entries in both sets consist of inputs (transcripts) and the desired outputs (summaries and insights), which were all manually validated by certified clinicians prior to the training. Algorithmically, the training process uses deep learning to update the base model's weights incrementally in a manner in which the total loss-defined as the total difference between the expected summaries and the model's predicted outputs-is reduced across training epochs. Epochs are cyclic intervals measured by when a complete set of training data is processed in its entirety during training. While more epochs typically create model behavior closer to the training set, a higher amount may produce overfitting and underperform during the subsequent validation set.

4. Validate the fine-tuned model with the validation set, and employ certified clinicians to examine outliers and re-tune the training set using the user interface of embodiments of the Aiberry invention. Upon the conclusion of training, the model is then used to generate notes and insights for the validation set's transcripts, and the loss of the models outputs are computed using, in an embodiment, the per-token perplexity calculation method. The entire training set is then augmented by certified clinicians using the user interface of embodiments of the Aiberry invention and the model is retrained, until the total validation loss reaches a satisfactory result, as defined, for example, by a ROUGE (Recall-Oriented Understudy for Gisting Evaluation) metric of at least 0.35. ROUGE is a set of metrics and a software package used for evaluating automatic summarization and machine translation software in natural language processing. The metrics compare an automatically produced summary or translation against a reference or a set of references (human-produced) summary or translation.

5. Create and deploy a custom Docker container as an AWS Lambda function in a virtual private cloud (VPC) that uses the Python Huggingface ecosystem and the trained model to infer results from a transcript in real-time, and return those insights and notes as a consumable JSON structured object (JSON or JavaScript Object Notation, is a text-based format for storing and exchanging data that's both human-readable and machine-parsable). The Docker container is an embodiment that is a Linux image minimized and optimized for virtualized inferencing architecture, such that it has reasonable cold-start times and the per-token processing speed allows for near real-time summary generation.

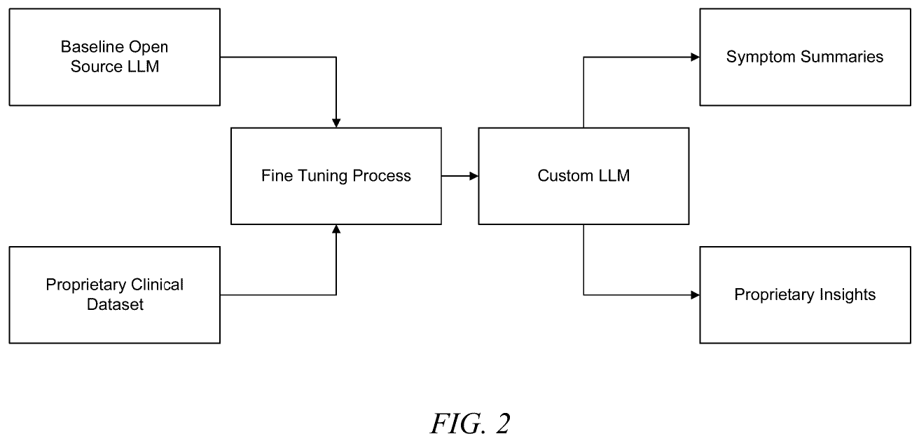

According to one or more embodiments, a custom LLM trained and deployed in the aforementioned manner can then process a proprietary clinical transcription as an input (a detailed description of which may be found in, for example, Intl. App. No. PCT/US23/24211) and produce a clinically useful and relevant summary of the presence or absence of symptoms, with clinically diagnostic details about those symptoms, as outlined in FIG. 2. Such embodiment also creates additional useful proprietary insights based on the transcript (for example, “last time happy” is “last Thursday at 6 PM”).

According to one or more embodiments, the structured response from the LLM is cached in the database and associated with the transcription. This data is then displayed in an interface to a practitioner or other user as illustrated in FIG. 1B; this interface displays preferably at least three key insights and a short transcript summary generated from the LLM. This view is designed primarily for clinicians, and therefore, the LLM model used will produce the summaries and insights most relevant for users in that context (practitioners). Additional models can address other users of the system, such as patients, by providing unique summaries trained specifically for them.

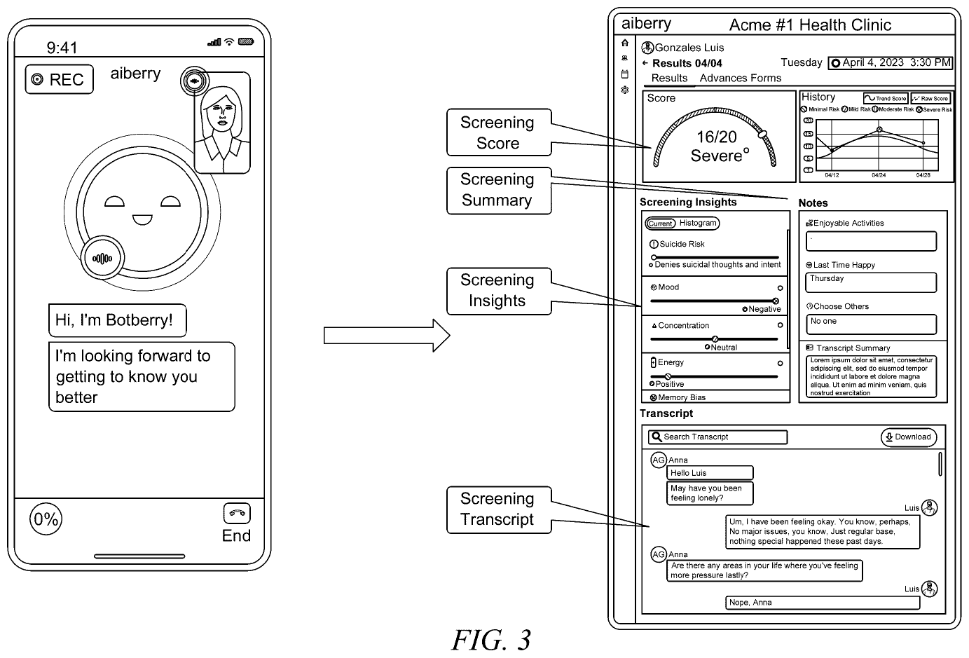

From a process flow perspective, the summary can be automatically created as part of the screening process and becomes available to the clinician as soon as the screening score becomes available. FIG. 3 illustrates the flow from conducting a screening to getting the screening outputs (score, transcript, transcript and summary). The process according to an embodiment may be executed as follows:

1. The member conducts an automated bot-assisted screening, and a clinical transcript generated from the audio stream (an exemplary detailed description of which may be found in, for example, International Application Serial No. PCT/US23/24211).

2. An inference process according to an embodiment transposes the raw transcript to a proprietary transcript structure (an exemplary detailed description of which may be found in, for example, International Application Serial No. PCT/US23/24211).

3. In one example of the embodiment, the inference process then invokes an API that is deployed on an AWS Lambda function that is hosting the LLM Docker container.

4. The LLM accepts the proprietary transcript as input and outputs a JSON object that includes the content for preferably at least three insights and the summary. The LLM also provides information regarding key phrases in the LLM, so as to indicate whether they are positive or negative so that the Aiberry UI can highlight them with specific colors, symbols or otherwise denote positivity, negativity or other parameters.

5. The structured output from the LLM then becomes an attribute of the overall screening object, and is stored, preferably in that database for quick retrieval.

6. If the LLM's version is updated to enhance its accuracy or features, an embodiment will reprocess all stored transcripts and resave the new outputs back to the database, ensuring that historical screenings use the most recently trained and capable LLM version. For example, if a model is trained to produce a new insight that did not exist previously, or if the ROUGE score has been reduced, this embodiment will ensure-through an automated process-that all previous screenings will be reprocessed to benefit from the updated behavior.

### Capability 3—Incorporating Summary Insights to Personalize the Screening Questions

This capability describes Aiberry's ability to utilize the LLM generated screening summary and insights to personalize the Botberry screening questions for each individual user.

To improve the screening experience, an embodiment utilizes information that was shared by the patient in past screenings and incorporates that past screening information into the questions that are being asked to make them more personalized or relatable to the patient and/or the clinician.

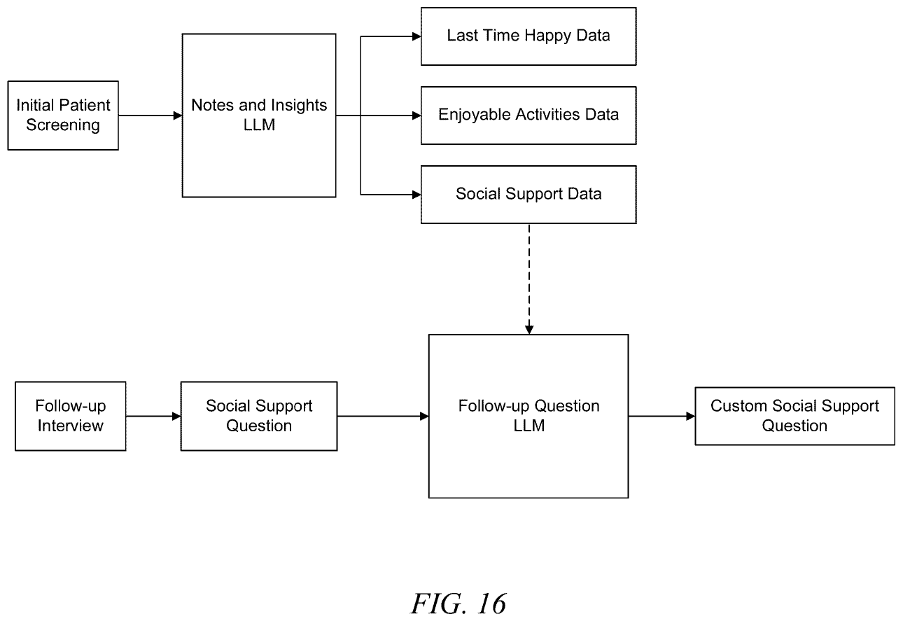

The LLM screening notes generator enables a user to capture and store such information as structured data in key-value storage rather than as plain text. By making such information readily accessible in, for example, a JSON document stored in a database, it can then be referred to in a subsequent screening, as FIG. 16 illustrates.

Each of the key insights from the LLM output can be attributed to a specific screening domain, for example “social support”. When the Aiberry system selects the questions to be asked in the screening, based on the specific domain, an algorithm will check if relevant insight for this domain exists from a previous screening by accessing the matching domain node in the previous notes' structured data.

If a relevant insight for this domain does not exist from a previous screening (such that there are no matching domain nodes in the previous notes' structured data), then the algorithm can use the generic notion of the screening question (e.g. “Tell me about interaction with family or friends”). Alternatively, if the algorithm detects a previous insight, then the screening process augments the question with generative AI to incorporate that insight in the question such that it will make it more contextual and personalized. For example, if the “social support” insight value is “boyfriend” then the question Botberry may ask could be “How is your relationship with your boyfriend?”

One or more embodiments includes a LORA fine-tuned version of a commercially-friendly LLM model (such as Facebook Llama 3.2), which creates these personalized questions and accounts for proper casing and plurals. In one exemplary embodiment, Aiberry fine-tuned this LLM by using a proprietary set of instruction prompts and paired completions to guide the model to generate these questions. In alternate embodiments, different proprietary or public instruction prompts and paired completions may be utilized to guide the model. The training set consists of the following:

1. An “instruction” prompt built from live clinical follow-up transcripts, which tells the LLM what the purpose of the question is (e.g. “To assess the levels of social support”), what the original generic question is (e.g. “Tell me about interaction with family or friends.”), what their previous answer was as recorded in the notes JSON, (e.g. “boyfriend”), and then instruct the LLM to formulate an ideal personalized question using the provided data intended.

2. A “completion” question that considers the instructions information, preferably written by a trained clinician using the Aiberry user interface, forms a private, proprietary dataset unique to Aiberry. The question is written by the certified clinician as a hypothetical ideal question to ask the patient in a follow-up assessment to elicit the most useful and relevant response from that specific patient.

Using the foregoing process steps, an assessment LLM model can thereby be trained to produce personalized questions that are substantially similar to what a clinician may ask with the information they would have had available to them about that patient.

According to an additional embodiment, prior to asking an assessment question, a mechanism exists to first run an inference with the assessment LLM to create a personalized question for the individual user as described above and then use a Text-To-Speech model (such as AWS Polly) to then present the personalized question to the user with as a spoken question.

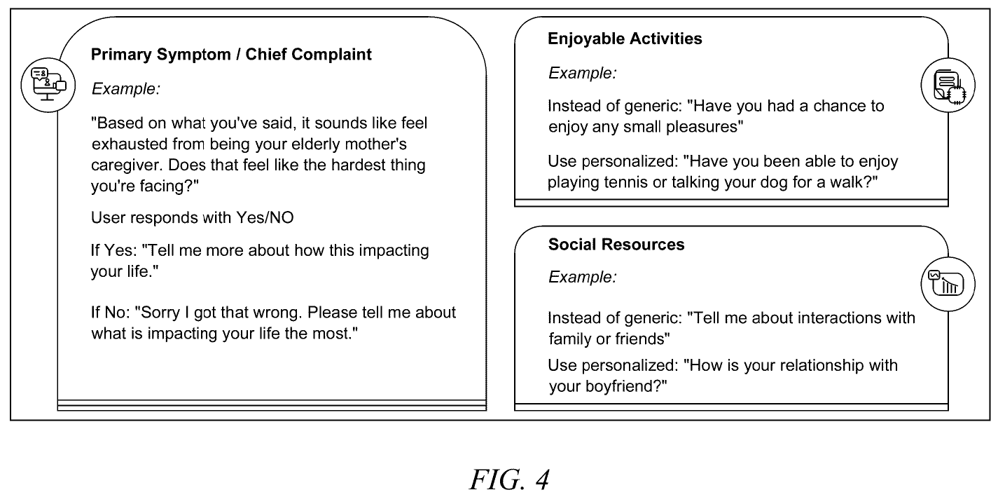

FIG. 4 illustrates some of the uses for personalized screening questions based on past screening insights and notes.

### Capability 4—Longitudinal Summaries

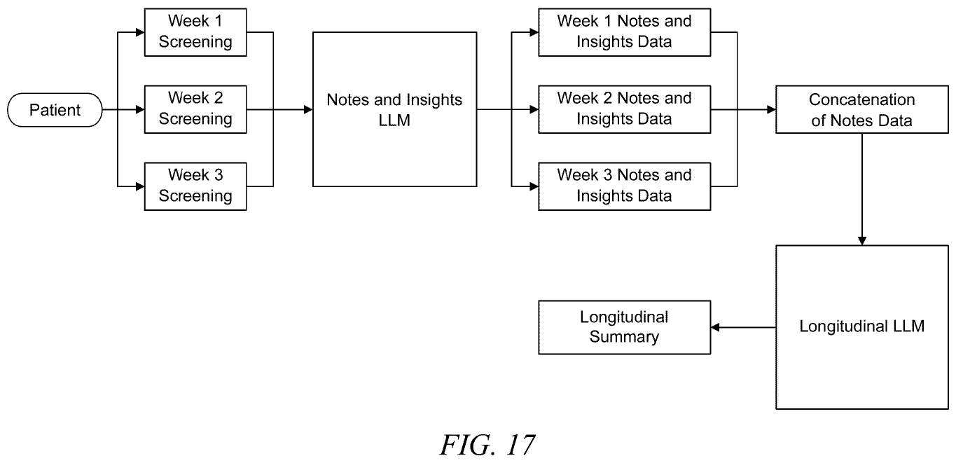

As illustrated in FIG. 17, this capability allows for the platform to consider multiple screening summaries and, through a custom LLM, create a high-level overview of a patient profile over their time of treatment. The output allows practitioners-by interacting with this embodiment-to discern a patient's high-level status by looking at a single, detailed longitudinal text summary of the patient's history. Because the longitudinal summary is to be based on an evolving data stream as new screening information is elicited and stored by the method and system, this patient overview will be dynamic and will evolve as the member's mental health situation evolves, keeping the overview for the practitioner constantly fresh and with up-to-date, current information.

One or more embodiments includes a proprietary time series database of patients' clinical assessments paired with LLM derived clinical summaries (see “Capability 2—Generating screening notes from a screening transcript”); all such records of patients with multiple assessments can be candidates for an anonymized training set.

A training set exists as an embodiment that contains patients' history of assessment data and the LLM generated notes; the notes data is formatted and concatenated into a single long text blob, each of which tells the sequenced history of the patient. For each historical record, preferably a certified clinician uses the interface of the Aiberry system to craft a summary or the notes that ideally captures the patient's state, progress and other relevant clinical insights, and this data is retained by the system and available for further processing according to the system and methods described herein.

One or more embodiments includes a longitudinal LLM that was trained using a historical training set and validated by certified clinicians to reach a minimum loss threshold. This longitudinal LLM, once deployed as an AWS Lambda function in a Docker container, will accept two or more historical records of a patient that have been formatted and concatenated into a single stringified JSON object as illustrated in “APPENDIX 3-Longitudinal JSON Summaries and Insights”. The longitudinal LLM is trained to accept this concatenated JSON structure and output JSON data containing insights and a clinically useful summary intended to capture the patient's historical record, as recorded through multiple screenings over time.

From a process flow perspective, the longitudinal summary can be automatically created as part of the screening process and becomes available to the clinician as soon as new clinical screening notes are generated. The process according to an embodiment—as FIG. 17 illustrates—may be done as follows:

1. The member conducts a Botberry screening that-through a previously described process-results in the generation of useful and relevant screening notes.

2. If multiple screening notes exist for the member, the screening data and accompanying notes are retrieved from the database and reformatted into a single stringified JSON object, as shown in APPENDIX 3.

3. The longitudinal LLM accepts the proprietary historical record as input and outputs a JSON object that includes the content for each summary and other key insights.

4. The structured output from the LLM then becomes an attribute of the overall patient object, and is stored in that database for quick retrieval.

According to one or more embodiments, the longitudinal summary will be presented to practitioners through a graphical interface on the patient's primary view, providing a high level text overview of the patient's status, accompanied by useful clinical insights (such as “condition improving”).

Capability 5—Continuous LLM Improvement Via Reinforcement Learning from Machine-Mediated Human Feedback

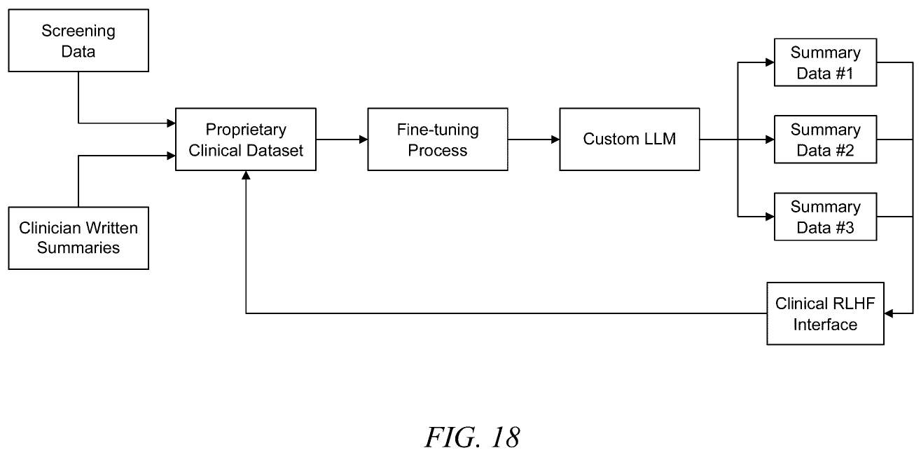

As illustrated in FIG. 18, this capability describes the Aiberry ability to create a continuous training pipeline, such that the LLM summarizers will incrementally produce more useful and relevant results, preferably as judged by certified clinicians and validated through a loss function¹. In turn, this set of automated training processes and graphical interfaces transforms the Aiberry platform from a simple assessment and tracking tool to a data collection and AI training engine. ¹ https://en.wikipedia.org/wiki/Loss_function

According to one or more embodiments, a continuous training pipeline exists for all custom trained AI models used by Aiberry; the custom depression LLM summarizer will serve as an example to illustrate this capability. As previously described by Capability 1 and 2, the initial LLM depression screening summary model has been trained on pairs of HIPAA-protected, proprietary live transcript data and summaries created by certified clinicians to be useful and relevant. This proprietary data is stored in a relational database-such as PostgreSQL—with additional fields such as timestamps and Boolean columns to flag the pairs as part of a training set or a validation set. PostgreSQL is an example of a free, open-source relational database management system (RDBMS). Relational databases allow for related but separate entities, such as Screenings and Summaries, to be stored in unique tables, and used to quickly retrieve data in an organized manner that may be composed of columns from multiple related tables.

As Aiberry conducts future screenings and creates matching summaries using the LLM from those transcripts, those potential training pairs of proprietary clinical data are candidates to be anonymized and aggregated into the training database in addition to the initial training and validation sets, as described by Capability 1.

According to one or more embodiments, clinicians can use a graphical user interface to view all potential candidates for training; such interface allows a clinician to compare the collected transcript to the automated LLM output and conduct Reinforcement Learning from Human Feedback (RLHF). The clinician can utilize the interface to rewrite a validated summary in a new column, and then flag the record as a training example, a validation example or otherwise ignore it. Such interface allows for the initial proprietary training set to be quickly expanded as new records enter the system, leveraging the existing LLM's output as a base for creating the ideal human generated summary (in the case that the LLM's summary was not ideal for the accompanying transcript).

For example, if a patient responds to an “Energy” question by saying “its ok, but I'm up and down”, the behavior of the LLM may produce a summary such as “The patient reports that their energy is okay and up and down” by default. A reviewing clinician can then produce a more useful and clinically relevant summary in the “summary_correction” column such as “The patient reports that their energy levels are fluctuating, but shows no signs of severe disorder”. The clinician can then toggle the example to be part of the training set. In this manner, unforeseen and unforeseeable gaps in the original training set are identified, completed, and retained by the system and method to be available for further processing.

One or more embodiments may include an automated program that detects any changes made to the training sets and retrains the relevant AI models. For each training set in the database, the following steps will be taken on one or more scheduled intervals:

1. A program will execute that will examine all database rows in the training set and the validation set, and determine if there have been any changes. For both the training and validation set, the program will concatenate the inputs (transcripts) and the desired outputs (summaries), and then compute the SHA-256 hashes of each set. SHA-256 stands for Secure Hash Algorithm 256-bit, which is a security hashing algorithm that can be used for general purposes. Hashing is the process of creating a finite and unique string of characters-in this case 64—from an input string; every conceivable input string should ideally produce its own unique hash. Therefore, if the hashes of the transcripts or summaries change from the previous run, it indicates that there have been changes to the training data.

2. If a change is detected, the training set will be retrieved from the database using an SQL statement with the appropriate column flags, and the resulting training pairs will be converted to a format appropriate for fine-tuning the base model, as previously described, utilizing the Python ecosystem (which will use a process similar to Appendix 2).

3. Once the fine-tuning has produced a new model, for each row in the validation set, the model will be used to produce a new summary and calculate the loss against the expected clinician-approved summary using the per-token perplexity calculation method; the output and loss of each row will be stored in a validation table of the PostgreSQL database. The final total loss of the fine-tuning job is equal to the average of each sequence loss.

4. For each fine-tuning job, a database record will be stored in a fine-tuning job table to remember its metadata-such as the total loss, the creation dates and information about its training and validation sets.

5. With the historical record of all validation pairs being tracked in the database for each fine-tuned LLM model version, the model “drift” can then be automatically calculated from one version to the next, with drift being defined as how adding, removing or editing any training pair impacts all other outputs against the validation set. The drift is calculated by the difference in the ROUGE score from one version to the next.

6. If no “drifting” outliers are found and the total model loss is equal or less than the previous, the new model will be deployed in a staging environment to an AWS Lambda function as a Docker container for quality assurance.

7. If excessive drift is found, the model version is queued and the training set will require clinician-directed re-tuning through interacting with the user interface of embodiments of the Aiberry invention, until it reaches a satisfactory loss and drift measurement on a subsequent training. By using an SQL statement with the validation table, any validation pair whose loss increased beyond some delta from the previous models' validation will be retrieved as outliers in the graphical interface.

The aforementioned automated training capabilities paired with the RLHF interface create a streamlined pipeline as an embodiment that allows the data collected from those using Aiberry to be continuously used to incrementally improve the Aiberry AI models.

### Stress and Burnout Visual Scales

One or more embodiments include a custom set of multiple visual scales that can be used to determine key measurements connoting stress and burnout. Stress and burnout may relate to or be caused by several factors, including e.g., employment, education or caregiving. Stress may be measured by the system using one or more visual scales for “Stress Load” and “Coping and Control”. Burnout may be measured by the system using visual scales for “Exhaustion and Depersonalization” and “Personal Accomplishment”.

The visual scales can be used to assess a patient's level of stress and burnout, and can be used in conjunction with Aiberry multi-modal Stress and Burnout screening.

One or more embodiments include an easy and relatable way to elicit inputs from patients concerning their perceived stress and burnout. The system and method described herein my utilize clinically validated self-report forms assessing stress (e.g., Perceived Stress Scale) and burnout (e.g., Maslach Burnout Inventory) are not free for commercial use (unlike PHQ-9 for depression), so these alternative scales were developed for this purpose. As such, and according to one or more embodiments, these custom forms provide effective scales available to the broader member base (outside of formal clinical settings).

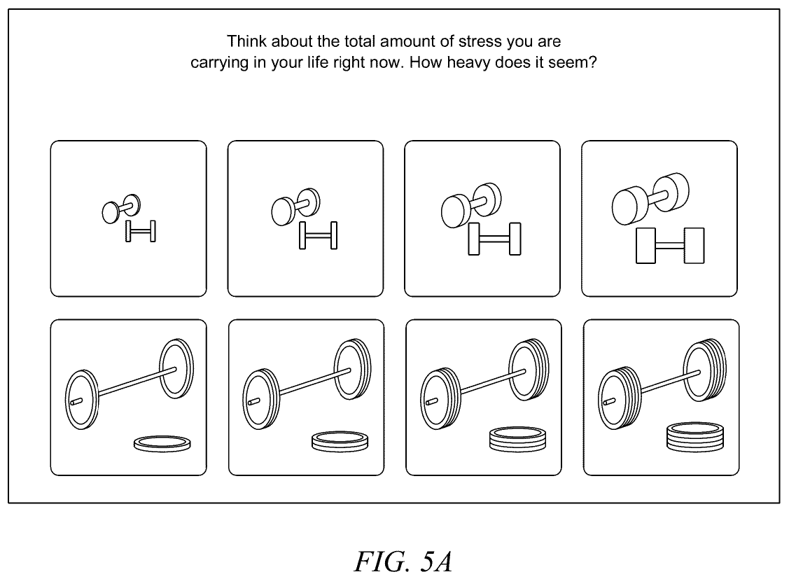

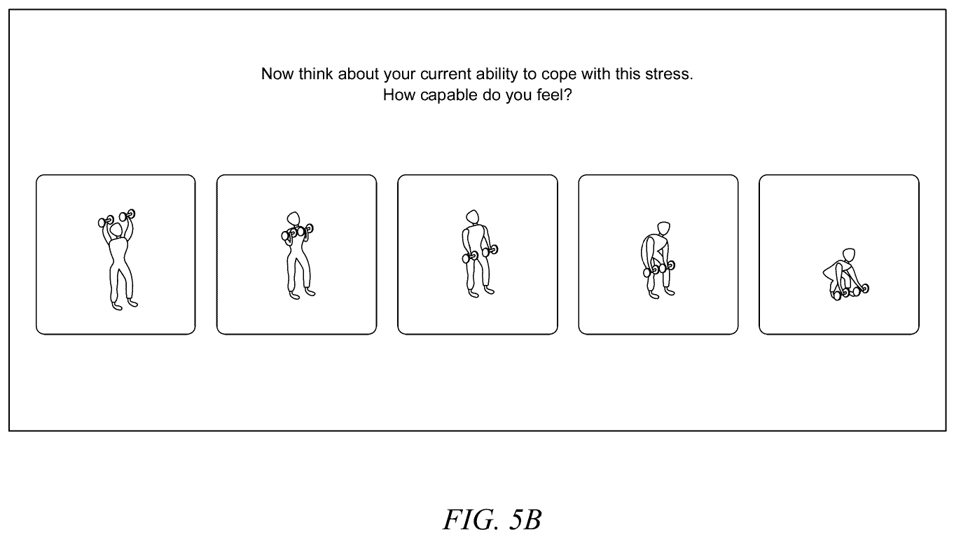

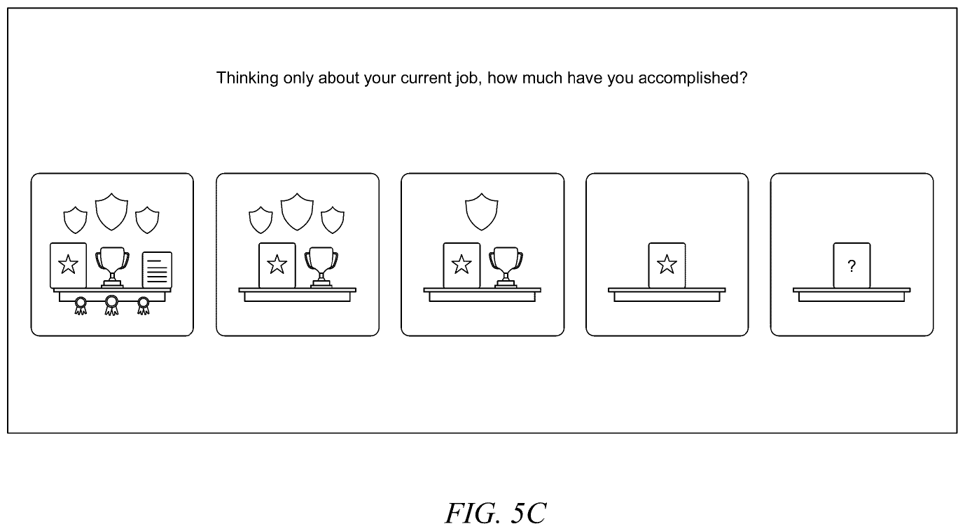

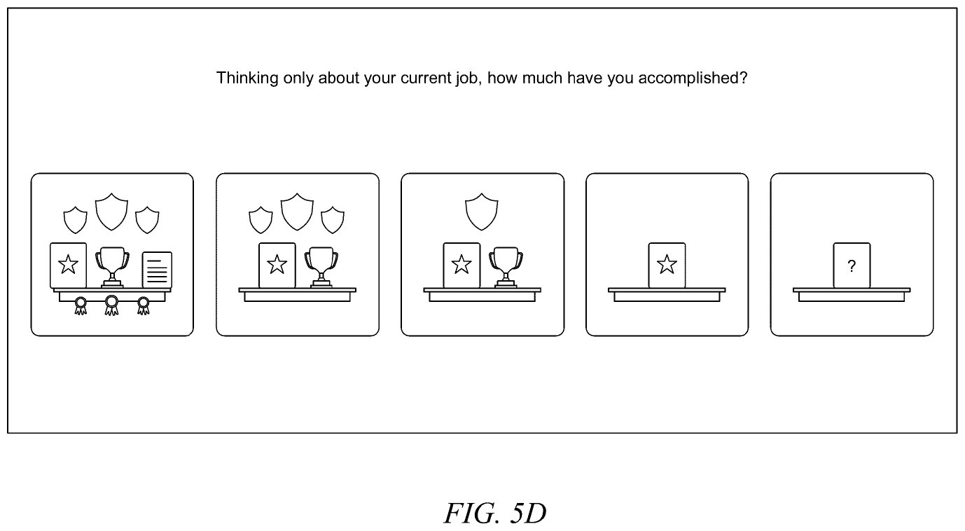

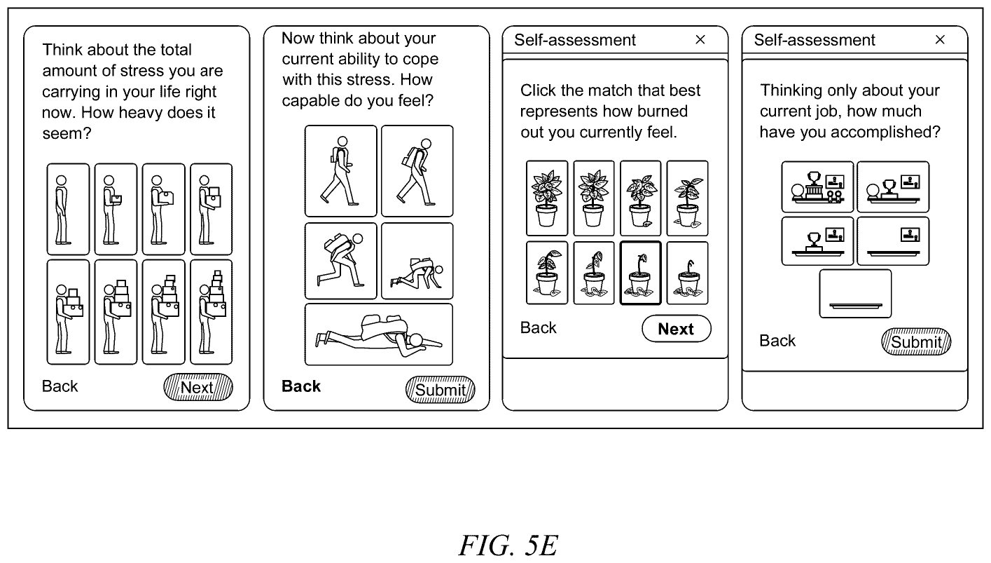

FIGS. 5A-5E illustrates some of the visual scales according to one or more embodiments. According to an embodiment, there are several visual scales. These are scales that are designed to correspond to specific questions that are being asked, as illustrated in FIGS. 5A-5E. Each visual scale includes clickable icons that the user can click once they determine which icon best represents how they subjectively feel in the context of the question asked.

### Capability 1—Stress and Burnout Digital Forms

One or more embodiments include a form for Stress and a form for Burnout. According to this embodiment, members can use those digital forms as a way of measuring and tracking their stress and/or burnout.

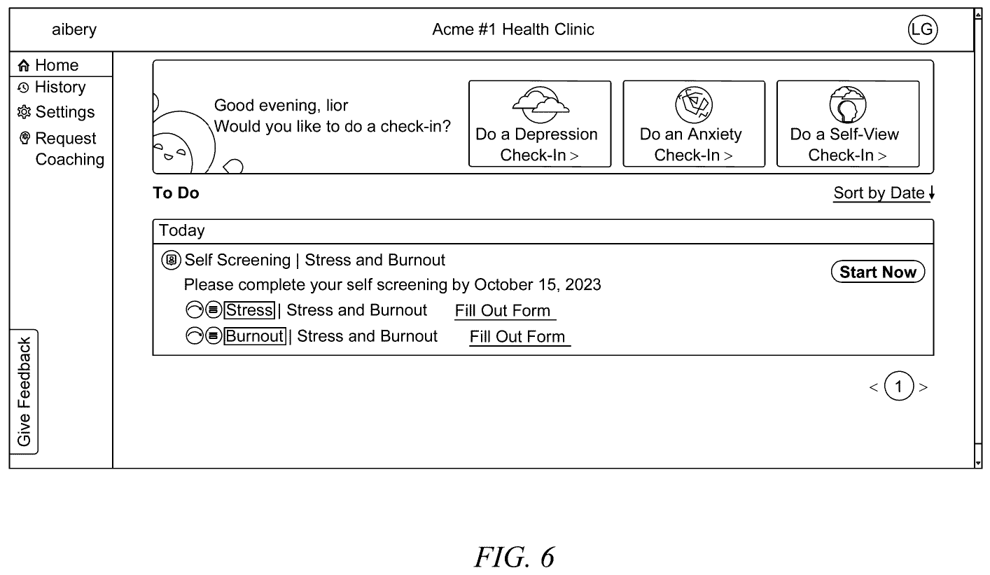

FIG. 6 demonstrates how the Stress and Burnout forms can be assigned to patients via self-assessment capability. This diagram illustrates how forms are assigned to patients by their practitioners and how the patients will view those pending Stress and Burnout forms in a member portal, all of which is accomplished through human interaction with one or more embodiments of the Aiberry invention. Once they press on the respective link, they will be presented with a digital form that will ask them two questions, which each have a respective visual scale mentioned in FIGS. 5A-5E as possible answers.

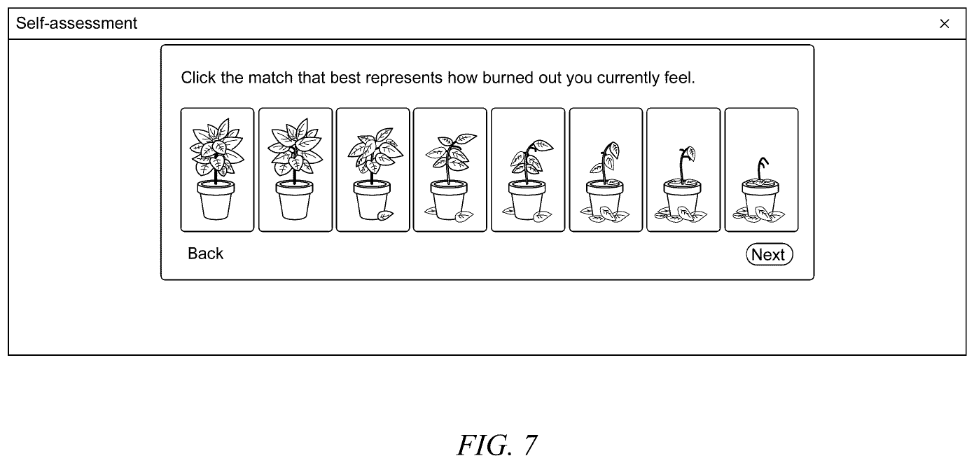

FIG. 7 illustrates how the visual scale can be incorporated into a Burnout digital form. Once a patient has pressed the link mentioned in FIG. 6, the digital form can be administered by Botberry. As illustrated in FIG. 7, a question will be asked and the respective visual scale will be presented. Each graphical icon on the visual scale is assigned a numeric value ascending from 0. For example, if the user selects the right-most image of the burnout scale depicted in FIG. 7, the score would be recorded as 7 indicating the maximum amount of burnout.

According to one embodiment of the invention, the user can choose one of the icons that best represents their subjectively perceived stress and burnout levels. Once they select the icon, they will press the “Next” button which will take them to the next question in the form. Once they are done, they can press a “Submit” button and the form is submitted for further processing and storage through the invention, and thereby, becomes part of the patient's historical record for tracking their mental health conditions.

### Multi-Modal Stress And Burnout Screening

According to one or more embodiments, a multimodal (Text/Audio/Video) artificial intelligence (AI) ensemble model to predict Stress and Burnout risk score is provided. While one or more embodiments covers mental disorders, and stress and burnout are not considered mental disorders per se, as such this distinct measurement creates an opportunity to increase the coverage of treatment. Stress and burnout could be considered as aggravating or precipitating factors for mental disorders, or in a broader category of mental health and wellness indicators.

In one embodiment, AI training data can be collected by conducting a study that involves, for example, several hundred participants. To create an AI training dataset for Stress and Burnout, the data collection mechanism must allow study participants to do two activities, as outlined below. First, they are to complete a Botberry-driven Stress and Burnout screening that consists of open-ended responses from the participants. Next, they are to complete digital forms consisting of visual scales that correspond to the same questions topics. The participants' multimodal (Audio/Text/Video) responses and visual scale scores form omnibus training pairs as an proprietary embodiment. Aiberry's unique clinical training set was additionally reviewed by clinicians to ensure there is a correlation between participant's answers and the visual scores, and the clinicians were generally in agreement with the self-reported scores. This clinically validated agreement is evidence that the visual scales (as described in “Stress and Burnout Visual Scales”) as a useful and effective way to numerically record a patient's perceived stress and burnout conditions.

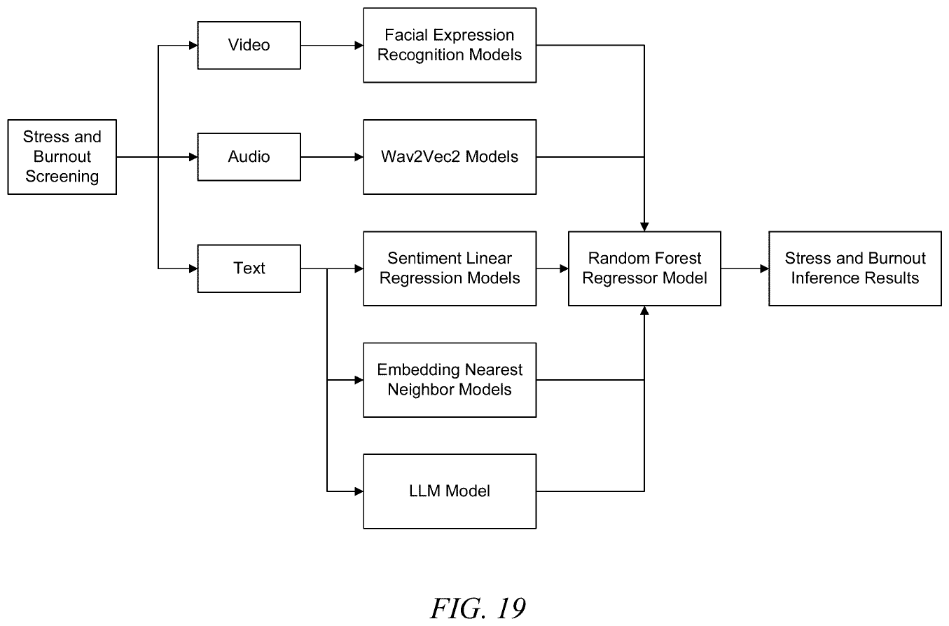

According to one or more embodiments, a custom collection of AI models are trained using the collected data to detect sets of features and insights specific to stress and burnout; such models are utilized according to a pipeline depicted in FIG. 19 to infer final risk scores. The AI model training is based on a comparison of open-ended responses (text/audio/video) that are collected during the Botberry screening and self-reported visual scale scores provided by the participants in the data collection activity. The following list of models are examples of the training approaches comprised by these embodiments:

1. For each open-ended question asked, a video AI model was trained. Facial Expression Recognition was employed by sampling frames from the video of a patient answering the question. Each frame image was processed using a Vision Transformer (ViT), and inferencing is then performed using an emotion detection model on the resultant vector. The emotion scores of all frames are averaged and paired with the participants' chosen score from the respective visual score to create a final training set. Regression models are then trained to predict the insight scores from the emotions detected on the patients' faces during their answers.

2. For each open-ended question asked, an audio AI model was trained. The video data in the training set was vectorized using Facebook's™ Wav2Vec2 model, which is a leading machine-learning model for the design of automatic speech recognition (ASR) systems. The vectors produced from the Wav2Vec2 model have been demonstrated to be able to accurately predict gender and emotion from audio files in public models. To train these proprietary models to predict domain insights, the patient's answer audio is vectorized and paired with the participants' chosen score from the respective visual score to create a final training set. After hyperparameter tuning, models can be trained to accept the vectorized audio of a patient answering a question, and thereby predict the corresponding score as a numerical output.

3. For each open-ended question asked, a linear regression model was trained to accept sentiment information. The transcribed responses in the training set are subjected to a sentiment analysis (AWS Comprehend, for example), and paired with the participants' chosen score from the question's respective visual score to create a final training set. The training results in a model that accepts sentiment information and uses it to predict the corresponding score as a numerical output.

4. For each open-ended question asked, custom nearest neighbor inference models are built using state-of-the-art (SOTA) transformer “embedding” models (such as “nomic-ai/nomic-embed-text-v1.5”). Embedding models are simply transformers that are trained to excel in specific tasks like clustering and classification, rather than for generating text; “embedding” is just another word for the vectorization of the tokens in a string. The embedding model is first used to create and store classification vectors for each transcribed question response, and then all vectors in the training set can be queried by searching a database (e.g. PostgreSQL with pgvector extension). To use this model, a patient's response is vectorized, and then an SQL statement is used to retrieve all other responses that are semantically similar to it in the “training set”. For example, if the participant's response is “I feel very good”, the vectorized embedding will be very close in vector space to a statement such as “I feel great”, and as such, all rows that are close to the input statement will be retrieved from the database. The nearest neighbors' recorded scores will be averaged in a weighted manner, based on their distance from the original statement, which is considered to be their semantic similarity to the original statement (as computed by the cosine similarity of the two vectors). As a result, these models use a clinician validated set in a database to compare against input statements and predict the corresponding score as a numerical output.

5. For the entire assessment, a custom text-based LLM model is built by fine-tuning commercially friendly models (such as Facebook's™ Llama 3.1 70b). First, the training set is augmented through an embodiment by processing the information with a private HIPAA compliant LLM to derive structured summaries, insights and contextually sentiment information. Next, an instruction prompt is constructed containing the transcription and the derived summaries and analysis information. The instruction prompt is then paired with a textual representation of the participants 4 visual scores as the desired completion to create the final training set; by combining all possible information about the assessment in a single context, the LLM has more information available at once when deriving the individual scores during the completion inferencing. After the model is trained and deployed, a participants' transcribed responses must be augmented and formatted identically to the training data, and then the model can be used to find the logarithmic probabilities for each score by utilizing a Monte Carlo algorithm during the inference process. To start, the formatted input is prepended to the initial completion string of “STRESS:”, and a single token is requested, such that the next token is the predicted score. After analyzing the probabilities of every considered permutation, a weighted average score can be predicted for each question as a numerical output.

6. Finally, for each open-ended question asked, an ensemble random forest regressor model is trained to predict the final scores that are shown to the users. The training set is composed of all numerical outputs of the aforementioned models paired with the participants' chosen score from the question's respective visual score. The random forest regressor is trained to find the most predictive features among the provided set from the audio, video, sentiment, nearest neighbor and generative LLM predictions, and use those predictive features to create decision branches. The resulting random forest model predicts a numeric score as its output.

Throughout the process of building these models, the collected data was strictly split (80/20%) into a training set and a smaller but different set for validation purposes that is proportionally reflective of the demographics of the participants and their scores. From a data collection perspective, one can make sure that the participant population is well-diverse (gender, age, ethnicity, etc.) to eliminate the risk of bias in the model, and thereby validate each model throughout training to ensure that no bias is introduced.

Turning to FIG. 19, one or more embodiments include an automated bot-driven (Botberry) screening process that will predict risk scores and insights (e.g., Rate of speech, Mood level, Energy level, etc.) relevant to stress and burnout. These predictions are made by asking relevant domain questions and analyzing the three modalities (i.e., text/audio/video) with the aforementioned collection of Stress and Burnout AI models.

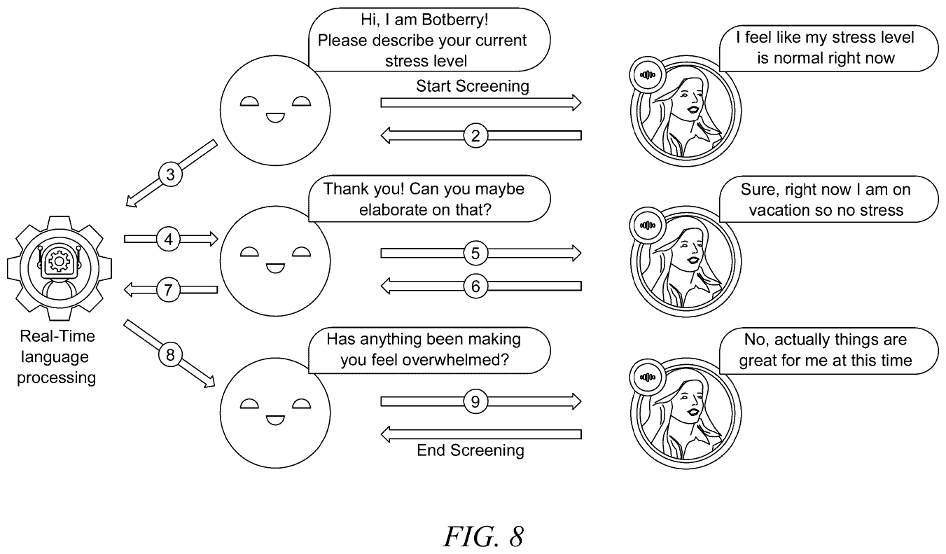

According to an embodiment, the Bot can analyze responses in real-time (as the screening is ongoing) and deliver relevant follow-up questions (as per an algorithm according to an embodiment). As an example, FIG. 8 illustrates what happens during a screening process according to an embodiment. Every time a response is received from the screening participants, a real-time language processing algorithm transcribes and analyzes the response according to clinically informed algorithms to determine what the next question should be. In this example, the engine “decided” (step 4) that it requires more information to make a confident prediction, and as such, presented (step 5) a follow-up question to the participant. Such algorithms to determine a follow-up question may simply consider the number of words in the participant's response, or instead the count of nearest neighbors in the database and consider if the confidence of a risk score inference based on the transcript is below some confidence threshold.

As shown in “APPENDIX 5-Stress And Burnout Follow-up”, a JSON data structure represents a portion of the Stress and Burnout script in an embodiment, and illustrates how the system utilizes the transcribed response to deliver such a follow-up instruction. When the transcript of question 2012 is sent to the server, a “postAction” is executed, in which the variable “_stress_load_followup” is set to the result of the “isUnderWordCountThreshold” equation. If the number of words in the spoken answer is under the threshold, “_stress_load_followup” is set to ‘true’. Thereafter, the next question to be asked must be determined from the remaining script. As illustrated in APPENDIX 5, the follow-up question 2013 is subject to a “realTime” filter, which specifies that it will only be shown if “_stress_load_followup” is ‘true’. By allowing the script to execute algorithms, set real-time screening variables, and react to those variables thereafter, this embodiment can exclude or include questions, and react specifically to what a patient is saying in real-time.

### Capability 1—Bot-Based Multimodal Stress and Burnout Screening

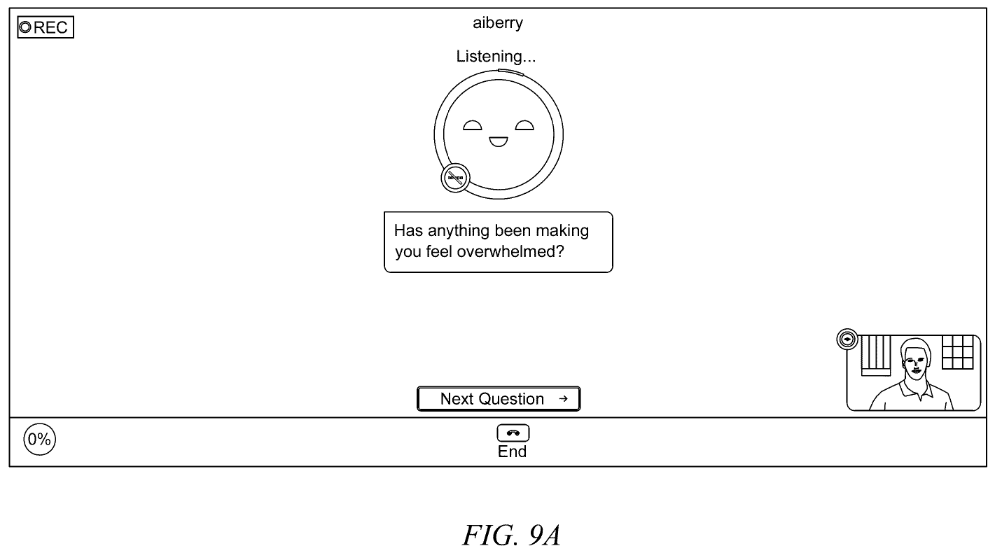

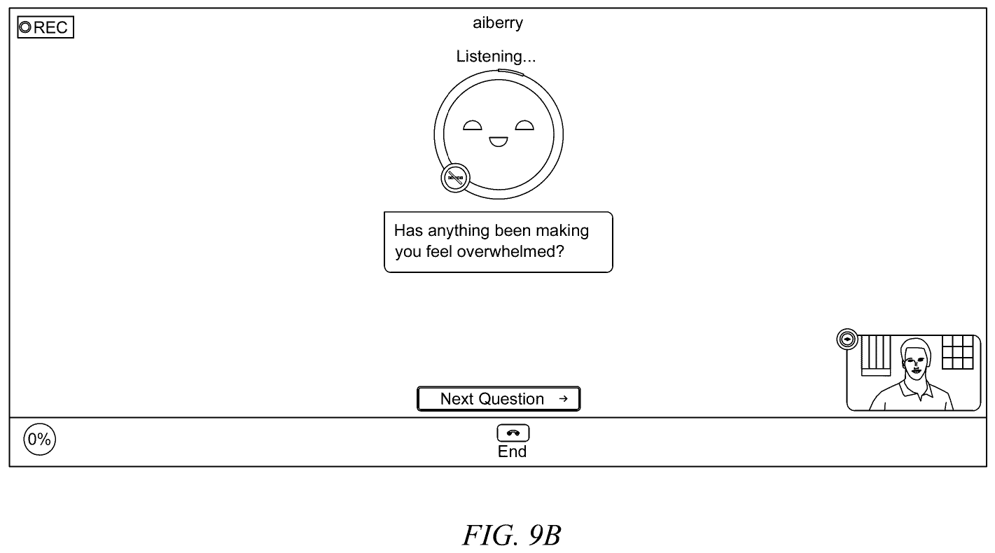

FIGS. 9A and 9B illustrate a Multimodal Stress and Burnout screening according to an embodiment. This screening process utilizes the same infrastructure and technology developed for other mental disorders, but uses a different question set and an alternative models for inferencing the Stress and Burnout domains (MULTIMODAL, AUDIO/TEXT/VIDEO, SCREENING AND MONITORING OF MENTAL HEALTH CONDITIONS). The addition of the Stress and Burnout screening illustrates how the totality of As shown in FIG. 18, embodiments of the invention create a consistent pipeline to collect clinical data, train AI models, conduct AI based assessments from those models and continuously improve those models based on RLHF, thereby creating the potential to assess and predict any conceivable clinical disorder.

According to one or more embodiments, the screening consists of open-ended questions to which the participant responses are analyzed. Each domain's question is randomly chosen from a larger bank of questions chosen by clinicians to elicit responses relevant for the target domain. As illustrated in “APPENDIX 6-Create Unique Experience for Each Screening”, this randomization routine will first remove questions asked in a participant's past screenings, such that each subsequent screening will be composed of a finitely unique permutation of questions. This embodiment increases engagement and reduces the participant's feeling of fatigue that would otherwise be aggravated by being presented with the same questions again and again.

### Screening Insights to Patient Actions—Recommendation Engine

Domains are logical groupings of potential symptomatology that contribute to a target disorder being accessed. For example, symptoms in “Mood”, “Social Support” and “Energy” domains are predictive indicators of depressive disorders.

According to one or more embodiments, a screening generates a set of domain specific insights in addition to a discrete numeric risk score. Those insights provide data to further dissect the risk score and understand the underlying contributing factors to the disorder being accessed, which is also a component of an “explainable Al” principle.

According to an embodiment, the risk score is the discrete numeric output that is derived from analyzing (inferencing) the Botberry screening transcript with the corresponding AI models. This is a number of a scale of 1-20 where:
1-5: Minimal risk
6-10: Mild risk
11-15: Moderate risk
16-20: Severe risk

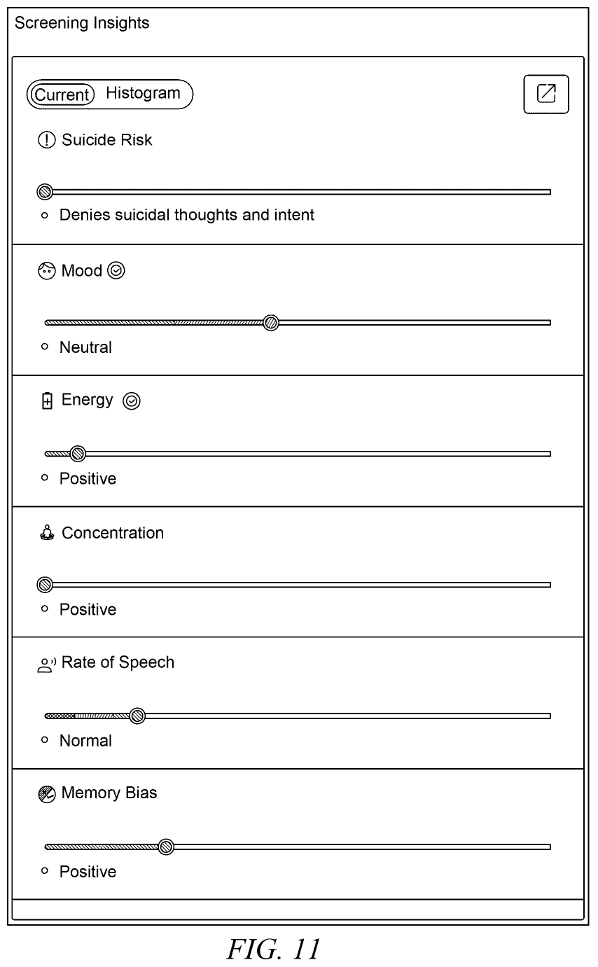

According to an embodiment, for each screening, the assessed risk score will be accompanied by a list of insights with a measurement on a positive to negative scale (1 to −1), such that those insights can be visualized as illustrated in FIG. 11. This information-considered in its entirety-serves as a detailed snapshot or “fingerprint” of a patient's current mental health condition respective to the assessed disorder (e.g. Depression).

According to one or more embodiments, the AI generated assessment information can now integrate to internal and external libraries of content (e.g., videos, articles, exercises), in such a manner that it “intelligently” matches resources specifically to the patient in an effort to improve impact and engagement. For example, if the screening result provides a very negative “Energy” insight (a value arbitrarily close to −1), the recommendation engine(s) can retrieve videos and resources meant to improve symptomatology associated with low energy disorders. There are two unique capabilities supported by one or more embodiments that can be utilized to support such recommendations: Retrieval Augmented Generative (RAG) searching and logic driven recommendation engines.

One or more embodiments include an ability to suggest a customized screening plan for a patient that can track and monitor specific areas for which we can then measure change over time and determine effectiveness. The numeric risk score and screening insights are submitted to rule-based engines that determine such screening plans, which operate according to specifications provided by certified clinicians.

One or more embodiments improve the targeting, utilization, and engagement of resource libraries available to patients. With longitudinal assessment tracking, there exists an ability to measure the impact of using the resource libraries by tracking improvements over time, and thereby developing AI models to predict the most effective resource for each unique assessment “fingerprint”.

One or more embodiments additionally utilizes the aforementioned longitudinal training data to provide companies with anonymized analytics data that demonstrates correlation—or lack thereof—between resources and progression impact.

### Capability 1—RAG Driven Recommendation Engine Based on Screening Insights

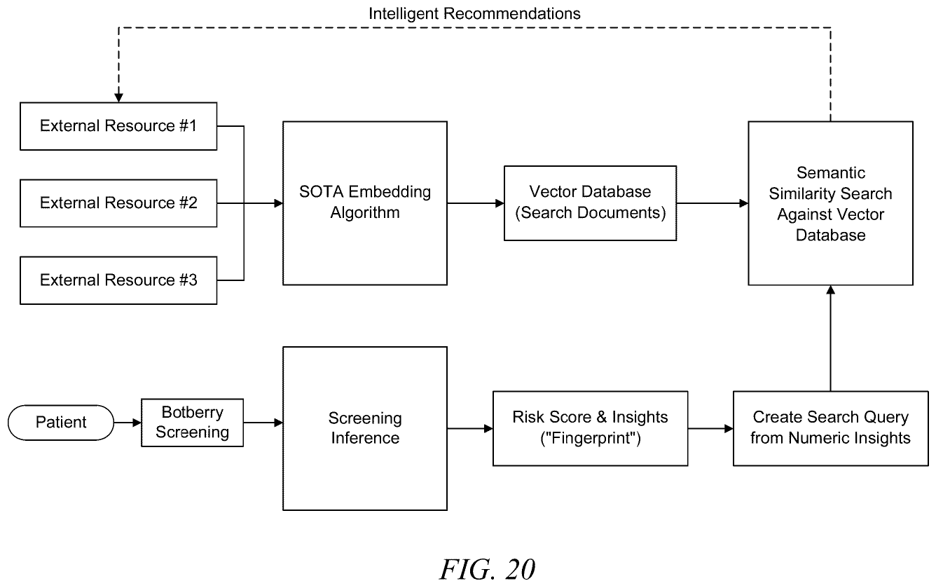

As shown in FIG. 20, this capability provides an AI driven mechanism for integrating a platform with external mental health libraries of resources and leverages score insights to make data-driven and accurate recommendations as to which resources to use.

There are many systems that offer external libraries such as videos, articles, exercises and therapeutic interventions. Those systems depend on the patient to navigate and consume the resources without sophisticated guidance and resources prioritization; Many of those systems suffer from low utilization, low engagement and no outcome based KPIs (rather, KPIs are based strictly on utilization and/or engagement without an integrated tracking mechanism incorporated into a wholistic platform).

As an alternative to burdening patients with a need to search for resources to treat their conditions, one or more embodiments leverages generative AI to produce a list of relevant resources that are specifically chosen to treat a patient according to their screening risk score and insights. In order to support this capability, a recommendation engine can be built to produce relevant and useful recommendations using the following steps:

1. For each resource in a library, convert the resource to a JSON text format. For example, videos can have their audio transcribed, and then the transcription would be submitted to an LLM to create an augmented JSON object containing the video title, its intent, and a summary description of what is discussed and shown in the video.

2. Once the resources have been normalized as structured textual data in JSON, that object will then be stringified into pure text and prepended with a term such as “search_document:” to facilitate Retrieval Augmented Generative (RAG) searching capabilities. The final strings will be vectorized using state-of-the-art (SOTA) GPT embedding models (such as “nomic-ai/nomic-embed-text-v1.5”) and stored in a search document database table associated with the resource.

3. As assessment information is generated from the screening process, a rule based system will analyze the output “fingerprint” (e.g. the risk score and insights) to identify the domains that contribute most strongly to the assessed disorder. Considering the aforementioned example regarding a negative “Energy” domain insight, a RAG based search string such as “search_query: how can I improve energy levels?” will be constructed from the analysis and vectorized with the chosen embedding model. The search query is then used in nearest-neighbor search performed with SQL on the resource library's database table, using cosine similarity to find the closest matching search documents, thereby retrieving the associated resources.

According to one or more embodiments, a capability emerges which leverages generative AI to allow for the delivery of relevant, targeted therapeutic interventions that specifically match the “fingerprint” of the patient's current mental health condition, which brings substantial value to the traditional approach of forcing patients to search manually across those resources for relevant help.

One or more embodiments includes training sets that contain the risk scores, domain insights and resources chosen as inputs, which are all paired with the n-th month delta in the patient's overall risk scores (representing improvement or deterioration). Using this training data, a Reranker model has also been trained from a commercially license friendly base model (such as “BAAI/bge-reranker-large”) that augments the RAG-based search results to deliver interventions that are not only relevant to a patient's condition, but historically most effective among the target population.

### Capability 2—Logic Driven Recommendation Engine Based on Screening Insights

This capability provides a logically controlled mechanism for integrating a platform with external mental health libraries of resources and leverages score insights to make data-driven and accurate recommendations as to which resources to use.

As previously described above, external resource libraries depend on the patient to navigate and consume the resources, and have minimal abilities to track long term outcomes and the effectiveness of such resources.

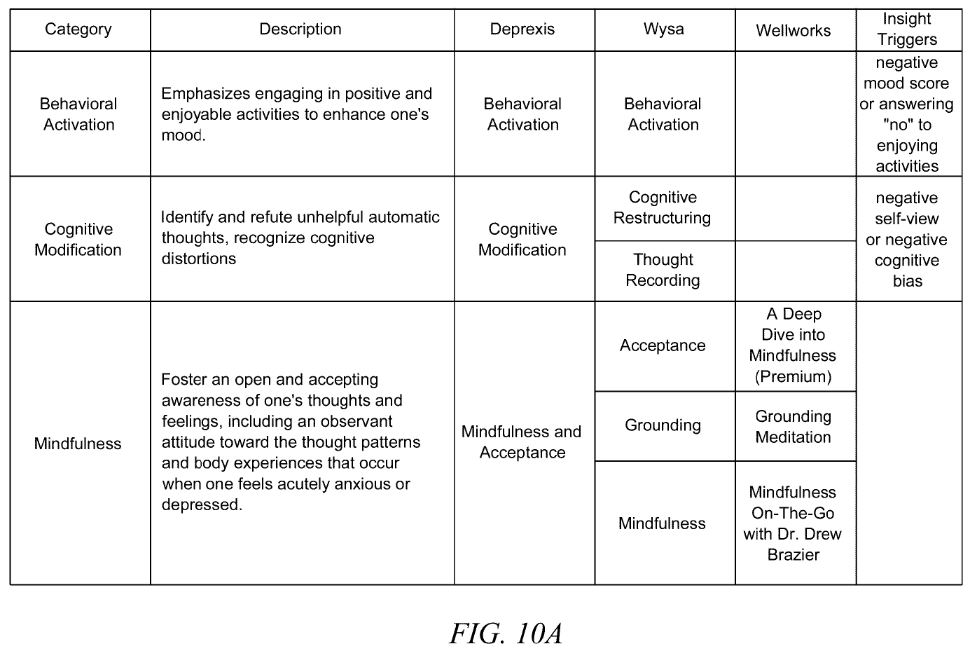

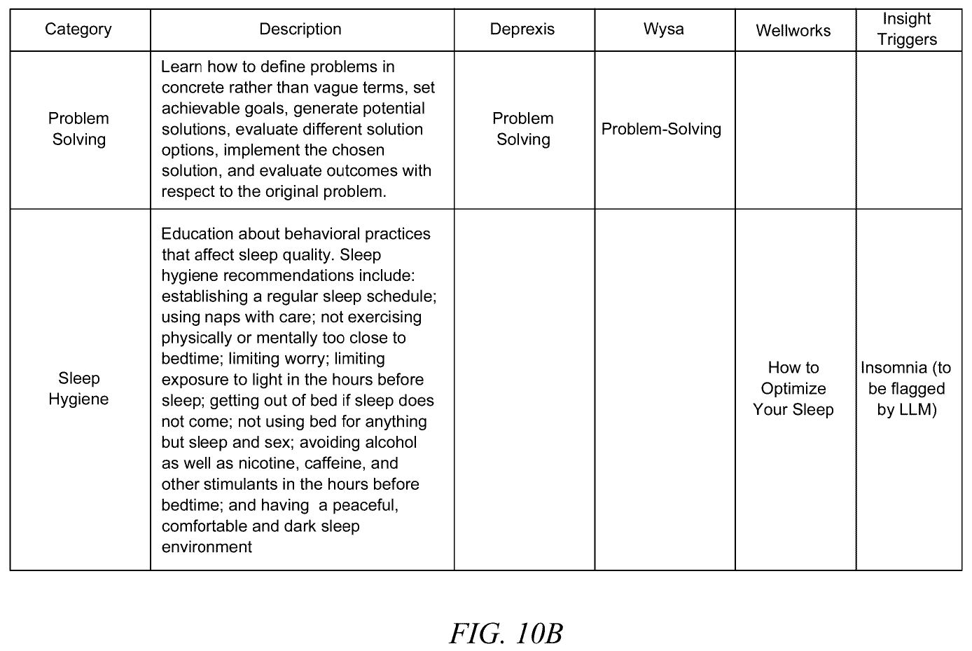

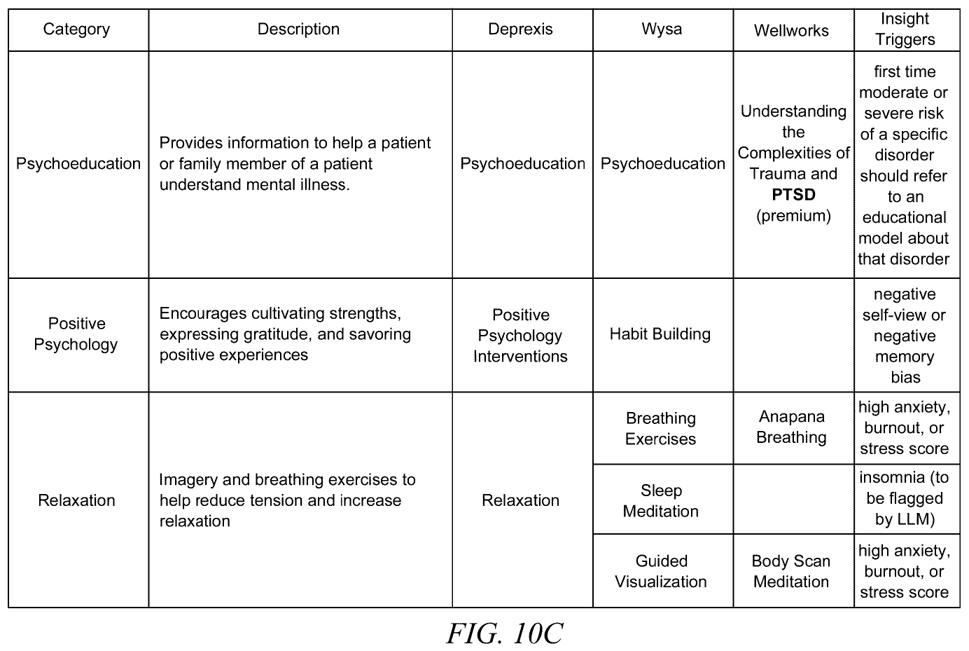

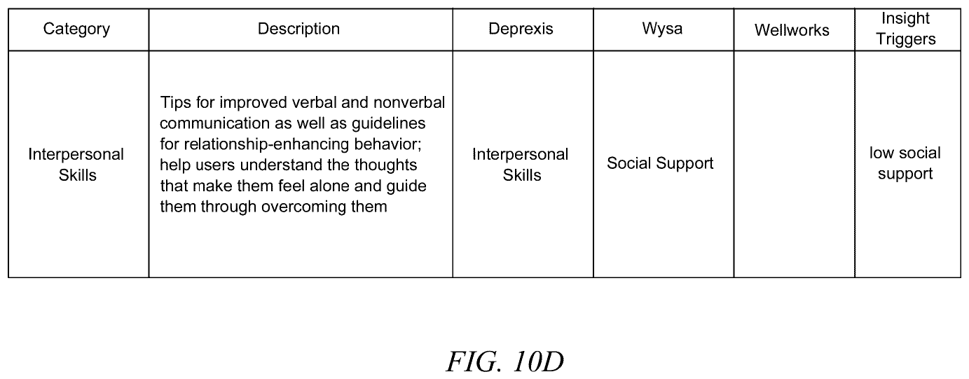

One or more embodiments include a map that can consistently connect a patient's assessment results to various disparate resource libraries using simple rules and categories. FIGS. 10A-10D are examples of such mapping to resources libraries and illustrates how such mapping is done by using examples of companies that own/offer such a library of resources (e.g., “Deprexis”, “Wysa”, “Wellworks”). The “category” column lists the category of the resources. The “Insight triggers” column relates to the Aiberry insights that are derived from the screening process according to one or more embodiments. As FIGS. 10A-10D illustrate, resources are organized by high level categories, and insight conditions can be used logically to trigger the recommendation of a specific resource.

As illustrated in FIG. 11, the Aiberry screening will generate a set of relevant insights in addition to the screening score, which are represented as numeric values between 1 and −1 inclusively. Accordingly, there are multiple insight categories such as “Mood”, “Energy”, “Concentration”, etc. Each of those insights are on a scale of Positive (Green to the left) to Negative (Red to the right). A positive insight value is indicative of minimal to no detected domain symptomatology; in other words, a score arbitrarily close to 1 indicates a healthy response. In the specific example in FIG. 11, “Energy” is very positive and indicative of no symptoms, while the “Mood” insight indicates the patient responded in a neutral or mixed manner to the domain question.

The “Insight triggers” in FIGS. 10A-10D are logical conditions that utilize the insight values, which are then encoded in a logic data structure (e.g. JSON logic) as an embodiment for each library. For example, the last column in FIG. 10D is “low social support”, which would be represented by a rule written in the chosen logic engine; thereafter, any input object evaluated that has a property “social_support” with a value less than 0 would thereby trigger this rule, and return the associated “Interpersonal Skills” resource.

One or more embodiments provides a mechanism that uses the logic rules to better determine the optimal resources for the patient to use. This capability requires that a data object consisting of the derived screening insights and other data to be inputted into the logic recommendation engine; the resultant output is the optimal resource for the targeted external library.

The recommendation engine can receive as inputs several data points such as:
1. Screening insights
2. Screening score
3. Resources that patient already completed in the past
4. Other recent available screening scores (multiple screening types)

According to one or more embodiments, a member can provide feedback as to the quality and relevance of a recommended intervention; this data is stored and used for improving the effectiveness of the recommendation engine. A linear regression model is used to determine correlations between resource usage, engagement and n-month outcomes. The correlation data is then used to inform future versions of the logic engine rules via using the user interface of embodiments of the Aiberry invention. When a user group provides their first baseline screening and uses a version of the recommendation engine, they form a unique starting cohort; the long-term outcomes of the subsequent cohorts are compared to the previous cohorts' outcomes validate the effectiveness of each iteration of the recommendation engines.

### Capability 3—Tracking Impact/Effectiveness of the Recommendation Engine

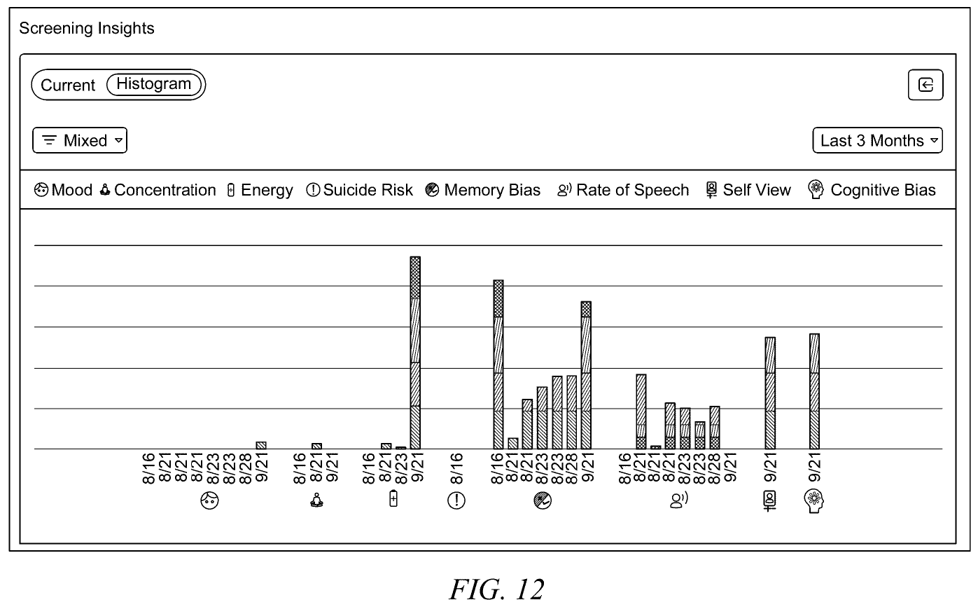

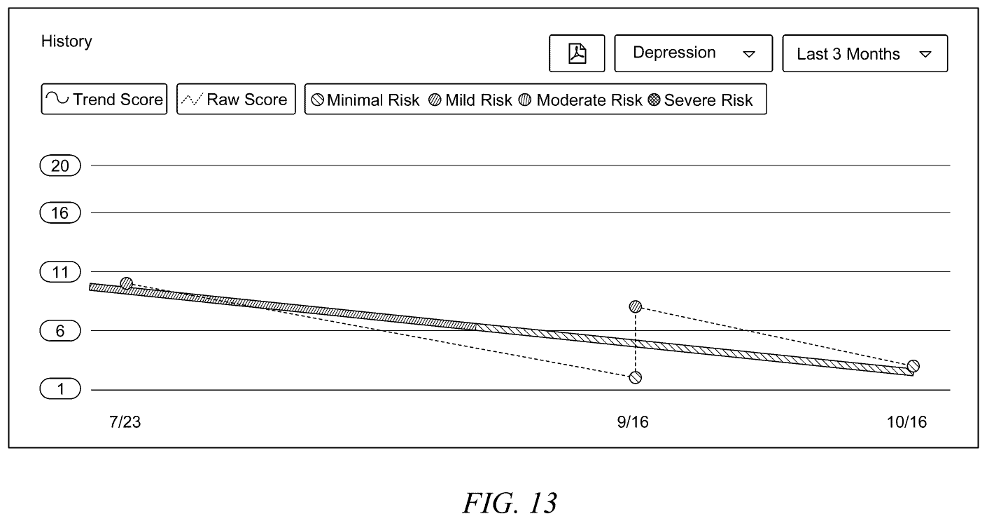

This capability works in conjunction with Capability 1 and 2 above. This capability enables tracking the various insights over time as illustrated in FIG. 12, as well as the overall risk score as illustrated in FIG. 13.

Through those graphs, the practitioners can track the changes in the specific insights and/or the overall score and infer the resources effectiveness by examining visual trends. Such correlations performed by certified clinicians validate whether the resources used contributed to any meaningful improvements among the cohort at large.

### Causal Inference Across Multiple Mental Health Screenings

One or more embodiments provides an overlay of risk-factor screenings and prognostic models to identify preceding or trailing cross impacts. For example: an acute rise in stress may be predictive of an increased risk for future mood and anxiety disorders. This ability enables organizations to push proactive interventions to disrupt or even correct negative health trajectories and prevent the onset or recurrence of mental illness.

As explained above in “Screening Insights to Patient Actions-Recommendation Engine”, screening insights can be integrated with internal and external libraries of content, e.g., videos, articles, exercises, and “intelligently” recommended to patients via RAG searching or logic based rules to improve mental health outcomes and engagement. In addition, based on causal inference enabled by the aforementioned embodiment, a practitioner can interact with the user interface of embodiments of the Aiberry invention to suggest or update a screening plan proactively for a patient cohort in response to discovering a predictive trend. Thereafter, that practitioner can continue to use the visualized overlay to track and monitor specific areas for which one can then measure change over time and determine effectiveness.

One or more embodiments allow practitioners to monitor individual patients or companies to get anonymized analytical data to show an overall organization or department situation.

One or more embodiments allow practitioners to interact with the user interface of the Aiberry invention to annotate and visualize external events alongside this analytical data, as to derive correlating preceding or trailing events that may impact—or be impacted by—the risk scores.

Combined, these embodiments facilitate detection and preventive treatment, offering a unique value to top-level users such as mental health providers or private organizations. For example, if an organization observes a sharp downward trend in “Mood” for an entire department, it may be causally inferred that this trend is predictive of the cohort's deteriorating mental health condition, as soon to be measured by the Depression risk assessment. Likewise, as an example, it may also be established and annotated that a rise in Depression scores correlates strongly with missed days at work, thereby impacting productivity and increasing health care costs for that organization. By utilizing this collection of embodiments together, an organization can proactively recommend interventions to the department to reverse the observed trend in “Mood”, and then monitor the long-term impacts of those interventions to calculate the return on investment in the cohort's mental health.

### Capability 1—Plotting Multiple Risk Scores on a Normalized Histogram

This capability allows the system to plot various risk scores on a single normalized histogram. The traditional screening tools used today use different scoring scales and, as such, plotting all of them on a single coherent normalized graph is not possible. Since one or more embodiments can use a unique method in which all the risk scores are normalized to a single proprietary score (e.g., 1-20 scale), various risk scores can be uniquely plotted on a single normalized scale in which the risk level of each is coherently and well compared to another. In developing the AI models, they may be trained on data collected from gold standard tools such as the PHQ's 0-27 scale or the GAD-7's 0-21 scale. Regardless of the collected training data, the Aiberry models are trained such that they are capable of being always normalized to the same risk score scale of 1-20, thereby enabling the aforementioned normalized graphing capability.

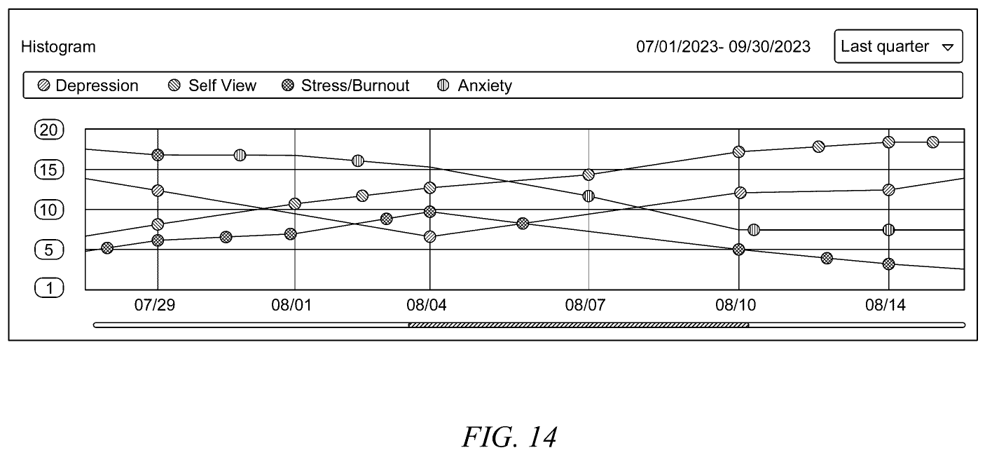

FIG. 14 illustrates this capability in which four different screening types (Depression, Self-View, Stress and Burnout, Anxiety) are plotted and compared on a single graph. Each line color represents a score “trend” (score over time) for each of those screening types.

### Capability 2—Causal Inference

Plotting various screening types on a normalized risk score provides a clinician or a patient with a longitudinal view of all screening results side-by-side and, as such, identify relationships among them and the overall long-term mental health condition of the patient. For example, there are well-documented correlations between negative self-view and depression disorders, as well as correlations between elevated stress and anxiety disorders. As such, according to one or more embodiments, there exists a visualization of early predictive risk scores that-if not treated-will likely have a causal effect on another mental disorder. In summary, this capability provides the ability to identify and track predictive trends over time and deliver preemptive interventions.

This capability works synergistically with “Screening Insights to Patient Actions-Recommendation Engine” as discussed above when considering the entire platform holistically; predictive trends can more easily be casually inferred by means of the visualizations, while the recommendation engine helps to determine the most effective interventions to deliver once those trends are identified.

### Capability 3—Annotations of External Events

While the normalized histogram data is composed of only data collected specifically through the Aiberry platform (e.g. depression assessments), such aggregated risk scores of an organization may have correlating events that are not collected by Botberry. For example, if a clinic introduces a new form of therapy and the impacted cohort shows a significant reduction in risk scores within some reasonable window, one could casually infer the introduction of the new therapeutic intervention is correlated to the changes, if and only if it was possible to visualize such external events overlaid on the normalized histogram.

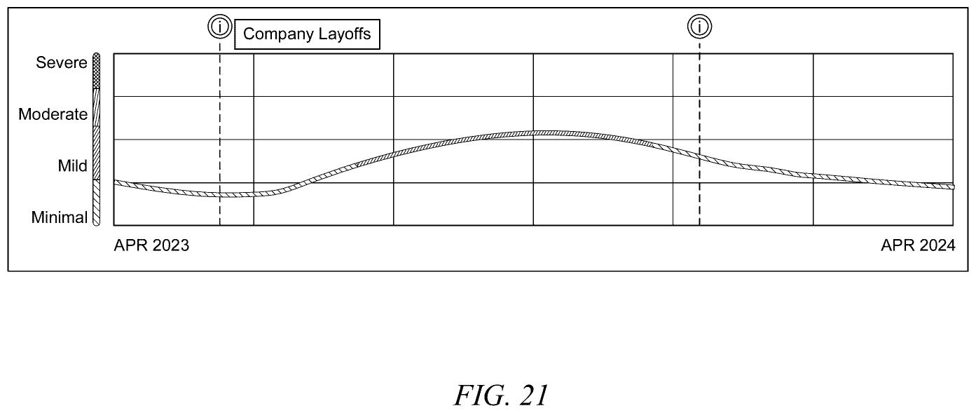

Alongside the risk score data, each organization is provided with a time series table as an embodiment in which an operator of an organization can annotate an external event by using the user interface of embodiments of the Aiberry invention. Annotation rows are composed of timestamps, titles, types and verbose descriptions. This data is simultaneously plotted as waypoints on top of the normalized histogram—as FIG. 21 illustrates-such that a user can draw causal associations to external events that may have leading or trailing associations with changes in risk scores. For example, an organization may annotate an event in which raises were given to a department; because such an event is now visualized on the normalized histogram, one could then track depression scores over the following weeks to determine if a pay raise is correlated to mental health outcomes. Over time, with enough annotations, an organization can discover which sets of pre-emptive events have the largest positive impacts on mental health conditions linked to performance and cost-savings.

### Outcome Analytics—Reliable Change

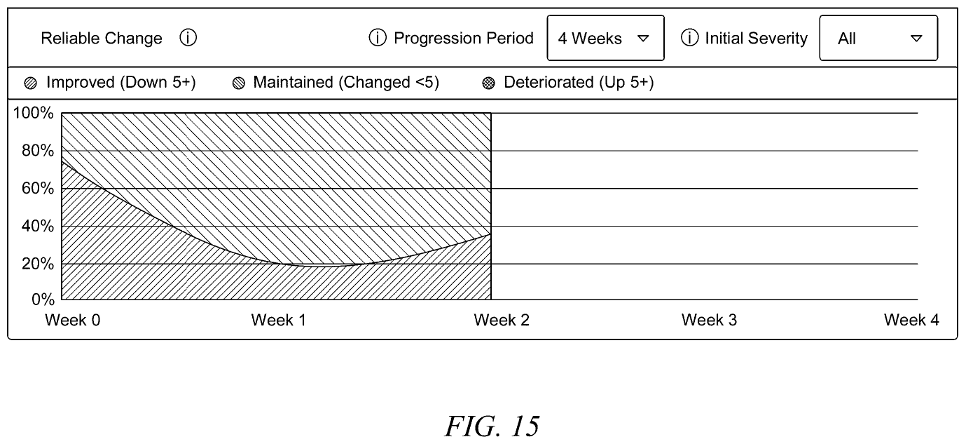

One or more embodiments includes a robust analytics module. The analytics module can provide different views to practitioners, one of which is the Reliable Change that is illustrated in FIG. 15. The Reliable Change graph visualizes the mental health (e.g., depression, anxiety) risk progression over a defined period of time. The unique approach of this graph is that it is not a snapshot over time but, rather, it identifies a population cohort and shows their risk progression over time from initial screening.

As such, one can measure the risk progress over time as a ‘normalized’ view since all patients' week 0 is considered their initial screening, and as such, every member of the cohort is on the same time scale regardless of the initial screening date. This graph creates a clear visualization of how the cohort members are doing after n-weeks of assessments, answering the question: “Are they doing better, the same or worse?”

One or more embodiments provide an ability to measure how a population of an organization is doing over time, which is a strong indication with respect to the effectiveness of their treatment.

### Capability 1—Tracking Risk Changes Over Time

The reliable change graph plots the meaningful improvements of outcomes (e.g., intake score+5) or deterioration (e.g., intake score −5) of a cohort of members over n-weeks who begin a baseline assessment within a specified time range.

According to one or more embodiments, the cohort is reduced to only those who also were subsequently assessed at the (N+/−20%*n) periods mark, with the nth week being selectable by the user. Missing weekly scores for individual patients are linearly interpolated between known data points, and all improvements and deteriorations are aggregated and plotted at weekly intervals. For example, if a member has a score of 10 at week 0 and a score of 12 at week 2, the missing week 1 datapoint will be interpolated as 11 ((10+12)/2).

According to one or more embodiments, a user can select a specific cohort of patients that have begun assessment tracking in the same interval and, as such, their initial screening is considered as week 0, and all following assessment scores are normalized to discrete weekly intervals (e.g. week 1, week 2, etc.). Otherwise, one would be mixing cohorts of people at different levels of treatment tenure, which will skew the results and reduce their usability in accessing treatment effectiveness.

As illustrated in FIG. 15, for example, the visualization indicates that after 2 weeks of treatment there is an observed improvement in the risk score, which is an indication of the effectiveness of the treatment. The data used to generate this graph may be screening results information collected from Botberry screenings. Every screening that is done in the system is captured in a database and available for visualization. This includes some key attributes such as:
Screening type
Screening score
Screening date
Screening insights

This information is then fed periodically into an analytics data lake, which is queried to create the Reliable Change view.

### Definitions

MEMBER: A USER who is limited to only using functionality associated with taking assessments, answering forms or viewing their past assessment history

PATIENT: A subset of a MEMBER, who uses the invention in a clinical setting, as directed by a health care PRACTITIONER.

PRACTITIONER: A USER who is limited to only using functionality associated with assigning screenings to PATIENTs and viewing the AI derived results.

USER: A superset of all user types.

This application is intended to describe one or more embodiments of the present invention. It is to be understood that the use of absolute terms, such as “must,” “will,” and the like, as well as specific quantities, is to be construed as being applicable to one or more of such embodiments, but not necessarily to all such embodiments. As such, embodiments of the invention may omit, or include a modification of, one or more features or functionalities described in the context of such absolute terms. In addition, the headings in this application are for reference purposes only and shall not in any way affect the meaning or interpretation of the present invention.

Embodiments of the present invention may comprise or utilize a special-purpose or general-purpose computer including computer hardware, such as, for example, one or more processors and system memory, as discussed in greater detail below. Embodiments within the scope of the present invention also include physical and other computer-readable media for carrying or storing computer-executable instructions or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the invention can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems or modules or other electronic devices. When information is transferred or provided over a network or another communications connection (either hardwired, wireless, or a combination of hardwired or wireless) to a computer, the computer properly views the connection as a transmission medium. Transmissions media can include a network or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. In some embodiments, computer-executable instructions are executed on a general-purpose computer to turn the general-purpose computer into a special purpose computer implementing elements of the invention. The computer executable instructions may be, for example, binaries, intermediate format instructions such as assembly language, or even source code.

According to one or more embodiments, the combination of software or computer-executable instructions with a computer-readable medium results in the creation of a machine or apparatus. Similarly, the execution of software or computer-executable instructions by a processing device results in the creation of a machine or apparatus, which may be distinguishable from the processing device, itself, according to an embodiment.

Correspondingly, it is to be understood that a computer-readable medium is transformed by storing software or computer-executable instructions thereon. Likewise, a processing device is transformed in the course of executing software or computer-executable instructions. Additionally, it is to be understood that a first set of data input to a processing device during, or otherwise in association with, the execution of software or computer-executable instructions by the processing device is transformed into a second set of data as a consequence of such execution. This second data set may subsequently be stored, displayed, or otherwise communicated. Such transformation, alluded to in each of the above examples, may be a consequence of, or otherwise involve, the physical alteration of portions of a computer-readable medium. Such transformation, alluded to in each of the above examples, may also be a consequence of, or otherwise involve, the physical alteration of, for example, the states of registers and/or counters associated with a processing device during execution of software or computer-executable instructions by the processing device.

As used herein, a process that is performed “automatically” may mean that the process is performed as a result of machine-executed instructions and does not, other than the establishment of user preferences, require manual effort.

Although the foregoing text sets forth a detailed description of numerous different embodiments, it should be understood that the scope of protection is defined by the words of the claims to follow. The detailed description is to be construed as exemplary only and does not describe every possible embodiment because describing every possible embodiment would be impractical, if not impossible. Numerous alternative embodiments could be implemented, using either current technology or technology developed after the filing date of this patent, which would still fall within the scope of the claims.

Thus, many modifications and variations may be made in the techniques and structures described and illustrated herein without departing from the spirit and scope of the present claims. Accordingly, it should be understood that the methods and apparatus described herein are illustrative only and are not limiting upon the scope of the claims.

APPENDIX 1
FINE-TUNING CODE
~/Dev/aiberry/transcript-summerizer/models/TranscriptSummary/finetune.py

```
import pandas as pd
import os
import shutil
from finetune_transcript_summarizer import finetune_llm
from huggingface_hub import login
from transformers import AutoModelForSeq2SeqLM, AutoTokenizer
from dotenv import load_dotenv
import sys
# Delete after testing
from datasets import load_dataset
def create_transcript_df(path_to_transcripts):
  df_transcripts = pd.read_csv(path_to_transcripts)
  # df_transcripts_prod = pd.read.csv(″finetune_transcript_summarizer/transcripts/
transcripts_from_pod_reduced.csv″)
  # df_transcripts_combined = pd.concat([df_transcripts,df_transcripts_prod])
  return df_transcripts
def push_to_hf(model_path):
  # Load finetuned model and tokenizer
  finetuned_summarizer = AutoModelForSeq2SeqLM.from_pretrained(model_path)
  finetuned_tokenizer = AutoTokenizer.from_pretrained(model_path)
  # Pusch finetuned model to aiberry Huggingface repo
  finetuned_summarizer.push_to_hub(f″aiberry/{model_path}″, private = True)
  # Push tokenizer to aiberry Huggingface repo
  finetuned_tokenizer.push_to_hub(f″aiberry/{model_path}″, private = True)
  return print(f″Finetuned model and tokenizer pushed to HuggingFace at repo
aiberry/{model_path}″)
def main(model_type):
  # Load HuggingFace Token and Log In (Enables pusch to hub)
  load_dotenv ( )
  login(os.getenv(″HUGGING__FACE_TOKEN″) )
  # Set arguments based on model_type
  if model_type == ″baseline″:
    transcript_path_arg = ″finetune_transcript_summarizer/transcripts/baseline_
transcripts.csv″
    model_path_arg = ″transcript-summary″
    summaries_dataset_arg = ″aiberry/clinician-transcript-summaries″
  if model_type == ″follow-up″:
    transcript_path_arg = ″finetune_transcripit_summarizer/transcripts/follow_up_
transcripts.csv″
    model_path_arg = ″transcript-summary-follow-up″
    summaries_dataset_arg = ″aiberry/clinician-transcript-summaries-follow-up″
  # Create one transcript data frame
  df_transcripts = create_transcript_df(transcript_path_arg)
  # Finetune the LLM
  finetuned_llm = finetune_llm(df_transcripts,
```

summaries_dataset_path_pipe = summaries_dataset_arg

′

finetuned_model_path = model_path_arg,
output_dir_pipe = model_path_arg,
num_epochs_pipe = 5, # Number of epochs trained loc

ally (Could increase if needed)

push_to_hub_pipe = False)

```
  print(″Finetuning completed″)
  # Push the model and tokenizer to HuggingFace
  push_to_hf(model_path_arg)
  # Remove the local version of the model and tokenizer
  shutil.rmtree(f″{model_path_arg}″)
  #print(transcript_summaries)
# On the command line follow this file with either ″baseline″ to finetune baseline
summarizer or
# ″follow-up″ to finetune follow-up summarizer
# >> python3 finetune.py ″follow-up″
main(sys.argv[1])
```

APPENDIX 2
LONGITUDINAL ISON SUMMARIES AND INSIGHTS
~/Dev/aiberry/docker/app/src/server/lib/EventLoop/Jobs/longitudal.json

```
[
  {
    ″date″: ″2024-11-19 17:43:57.525878+00″,
    ″summary″: [
      {
        ″insight″: ″not discussed″,
        ″severity″: ″Neutral″,
        ″insight_type″: ″last_time_happy″
      },
      {
        ″insight″: ″Hiking and Running″,
        ″severity″: ″Neutral″:,
        ″insight_type″: ″activities_enjoyed″
      },
      {
        ″insight″: ″their family″,
        ″severity″: ″Neutral″,
        ″insight_type″: ″close_others″
      },
      {
        ″insight″: {
          ″summary_text″: ″The participation’s mood has been great. They are
  enjoying activities as much as they have in the past. They report their
sleep is good, their self-confidence is high, and suicidal ideation was
not assessed.″
        },
        ″severity″: null,
        ″insight_type″: ″transcript_summary″
      }
    ]
  },
  {
    ″date″: ″2024-10-23 17:42:51.615874+00″,
    ″summary″: [
      {
        ″insight″: ″today″,
        ″severity″: ″Minimal″,
        ″insight_type″: ″last_time_happy″
      },
      {
        ″insight″: ″running, walking, and talking″,
        ″severity″: ″Neutral″,
        ″insight_type″: ″activities_enjoyed″
      },
      {
        ″insight″: ″nobody″,
        ″severity″: ″Neutral″,
        ″insight_type″: ″close_others″
      },
      {
        ″insight″: {
          ″summary_text″: ″The participant’s mood has been pretty great.
They are enjoying activities as much as they have in the past. They report
their sleep is good, their self-confidence is wonderful, and suicidal ideation
was not assessed.″
        },
        ″severity″: null,
        ″insight_type″: ″transcript_summary″
      }
    ]
  }
]
```

APPENDIX 3
STRESS AND BURNOUT FOLLOW-UP
~/Dev/aiberry/docker/app/scripts/Stress-And-Burnout.json

```
[{
    ″id″: 2012,
    ″rank″: 2,
    ″text″: ″How much strain are you under this week?″,
    ″time″: 60,
    ″type″: 4,
    ″label″: ″Stress_Burnout_Stress_Load″,
    ″postActions″: {
      ″evaluate″: {
        ″_stress_load_followup″: {
          ″equation″: ″isUnderWordCountThreshold″,
          ″variable″: {
            ″isUnderWordCountThreshold″: {
              ″algorithm″: ″underScreeningWordCountThreshold″
            }
          }
        }
      }
    }
  },
  {
    ″id″: 2013,
    ″rank″: 3,
    ″text″: ″${FOLLOWUP::SCREENING}$″,
    ″time″: 60,
    ″type″: 4,
    ″label″: ″Stress_Burnout_Stress_Load″,
    ″filter″: {
      ″realTime″: {
        ″conditions″: {
          {
            ″type″: ″variable″,
            ″condition″: {
              ″_stress_load_followup″: true
            }
          }
        ]
      }
    }
}]
```

APPENDIX 4
CREATE UNIQUE EXPERIENCE FOR EACH SCREENING
~/Dev/aiberry/docker/app/src/server/lib/rpc/screening.ts

```
if (!appointment.config.script) {
  // Retrieve the screening script from the database if
  // not provided using the appointment type data
  const script =
    alternativeScript ??
    (await this.getScript ({
      appointment,
      session,
    }));
  // Retrieve a list of all the past questions
  // that have previously been asked of the patient
  const pastQuestions = await this.getPastQuestions ({
    patient: appointment.subject,
    assessmentType: appointment.assessmentType,
    ...(appointment.assessmentType === Assessment.Type.STRESS_BURNOUT
      ? { lookback: 10 }
      : { daysSince: script.daysSince }),
  });
  // Remove the previously asked questions, and
  // randomly pick 1 of the remaining questions per domain
  const filteredScript = this.removeMultipleQuestions(
    script.questions,
    pastQuestions,
  };
  // Insert any relevant variables and follow-up
  // questions in the remaining script questions
  const finalScript = this.interpolateTemplatedQuestions(filteredScript);
```

What is claimed is:

1. A computer-implemented method, comprising the steps of:
receiving user responses over a network from a recorded mental health screening interview;
curating HIPAA protected health information from the screenings into unique artificial (AI) training datasets intended to produce unprecedented diagnostic behavior;
training custom Large Language Models (LLM) from such proprietary clinical datasets composed of such user responses to create clinically useful insights;
generating clinically useful insights from the user screenings for diagnostic purposes by employing such LLMs;
recommending useful interventions from external libraries to users by analyzing the generated insights and using AI searching algorithms;
improving the clinical accuracy of such insights LLMs by means of a graphical user interface supporting Reinforcement Learning through Human Feedback as part of a continuous AI clinical training engine;
and generating clinically useful longitudinal mental health summaries by using a custom LLM to analyze the aforementioned generated insights.

2. The method of claim 1, further comprising the steps of:
assessing Stress and Burnout by means of a clinically-validated visual scale presented to users through a graphical questionnaire;
training AI models to accurately predict Stress and Burnout from clinically gathered data;
predicting Stress and Burnout risk scores and insights by analyzing a video received over the network of a user answering questions and using the trained AI models.

3. The method of claims 1 and 2, further comprising the steps of:
displaying in a graphical display reliable change over time, to visualize the effectiveness of treatment for a single cohort over time;
displaying in a graphical display the normalized mental health risk scores for disparate disorders to allow for causal inference (i.e. the identification of correlations between disorders);
and through a graphic interface, graphing annotations of potentially predictive events on top of such risk scores to allow causal inference and to aid in proactive intervention delivery.
